(19) United States
(12) Patent Application Publication
(10) Pub. No.: US 2008/0153873 A1
(43) Pub. Date: Jun. 26, 2008

(54) DIHYDROBENZOFURANYL DERIVATIVES AND METHODS OF THEIR USE

(75) Inventors: Puwen Zhang, Audubon, PA (US); Joseph Peter Sabatucci, Collegeville, PA (US); Eugene Anthony Terefenko, Center Valley, PA (US); Eugene John Trybulski, Huntingdon Valley, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/954,469

(22) Filed: Dec. 12, 2007

Related U.S. Application Data
(60) Provisional application No. 60/869,646, filed on Dec. 12, 2006.

(51) Int. Cl. A61K 31/4709; C07D 405/04; A61K 31/4045; A61K 31/4184; A61P 1/00; A61P 25/02; A61P 25/00; A61P 25/24; A61P 13/00; C07D 307/83; A61K 31/343

(52) U.S. Cl. 514/314; 548/464; 514/414; 549/466; 514/470; 546/167; 548/305.1; 514/387

(57) ABSTRACT

The present invention is directed to dihydrobenzofuranyl derivatives of formula I:

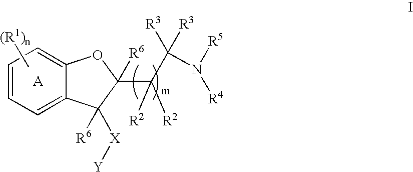

or a pharmaceutically acceptable salt thereof, which are monoamine reuptake inhibitors, compositions containing these derivatives, and methods of their use for the prevention and treatment of conditions, including, inter alia, vasomotor symptoms sexual dysfunction, gastrointestinal disorders and genitourinary disorder, depression disorders, endogenous behavioral disorder, cognitive disorder, diabetic neuropathy, pain, and other diseases or disorders.

### CROSS-REFERENCE TO RELATED APPLICATIONS

This application claims the benefit of U.S. Provisional Application No. 60/869,646, filed Dec. 12, 2006, the contents of which are incorporated herein by reference in their entirety.

### FIELD

The present invention relates to dihydrobenzofuranyl derivatives, which are monoamine reuptake inhibitors, compositions containing these derivatives, and methods of their use for the prevention and treatment of diseases or disorders, including, vasomotor symptoms, depression disorders, endogenous behavioral disorders, cognitive disorders, sexual dysfunction, or pain conditions in particular, vasomotor symptoms.

### BACKGROUND

Vasomotor symptoms (VMS), referred to as hot flushes and night sweats, are the most common symptoms associated with menopause, occurring in 60% to 80% of all women following natural or surgically-induced menopause. VMS are likely to be an adaptive response of the central nervous system (CNS) to declining sex steroids. To date, the most effective therapies for VMS are hormone-based treatments, including estrogens and/or some progestins. Hormonal treatments are very effective at alleviating VMS, but they are not appropriate for all women.

VMS are caused by fluctuations of sex steroid levels and can be disruptive and disabling in both males and females. A hot flush can last up to thirty minutes and vary in their frequency from several times a week to multiple occurrences per day. The patient experiences a hot flush as a sudden feeling of heat that spreads quickly from the face to the chest and back and then over the rest of the body. It is usually accompanied by outbreaks of profuse sweating, and may sometimes occur several times an hour, and it often occurs at night. Hot flushes and outbreaks of sweats occurring during the night can cause sleep deprivation. Psychological and emotional symptoms are also observed, such as nervousness, fatigue, irritability, insomnia, depression, memory loss, headache, anxiety, nervousness or inability to concentrate and are caused by the sleep deprivation following hot flush and night sweats (Kramer et al., In: Murphy et al., 3^{rd} Int'l Symposium on Recent Advances in Urological Cancer Diagnosis and Treatment-Proceedings, Paris, France: SCI: 3-7 (1992)).

Hot flushes may be even more severe in women treated for breast cancer for several reasons. Many survivors of breast cancer are given tamoxifen, the most prevalent side effect of which is hot flush, and many women treated for breast cancer undergo premature menopause from chemotherapy. Women with a history of breast cancer have also generally been denied estrogen therapy because of concerns about potential recurrence of breast cancer (Loprinzi, et al., Lancet, 2000, 356(9247): 2059-2063).

Men also experience hot flushes following steroid hormone (androgen) withdrawal. This is true in cases of age-associated androgen decline (Katovich, et al., Proceedings of the Society for Experimental Biology & Medicine, 1990, 193(2): 129-35) as well as in extreme cases of hormone deprivation associated with treatments for prostate cancer (Berendsen, et al., European Journal of Pharmacology, 2001, 419(1): 47-54. As many as one-third of these patients will experience persistent and frequent symptoms severe enough to cause significant discomfort and inconvenience.

The precise mechanism of these vasomotor symptoms is unknown but generally is thought to represent disturbances to normal homeostatic mechanisms controlling thermoregulation and vasomotor activity (Kronenberg et al., “Thermoregulatory Physiology of Menopausal Hot Flashes: A Review,” Can. J. Physiol. Pharmacol., 1987, 65:1312-1324).

The fact that estrogen treatment (e.g. estrogen replacement therapy) relieves the symptoms establishes the link between these symptoms and an estrogen deficiency. For example, the menopausal stage of life is associated with a wide range of other acute symptoms as described above and these symptoms are generally estrogen responsive.

It has been suggested that estrogens may stimulate the activity of both the norepinephrine (NE) and/or serotonin (5-HT) systems (J. Pharmacology & Experimental Therapeutics, 1986, 236(3) 646-652). It is hypothesized that estrogens modulate NE and 5-HT levels providing homeostasis in the thermoregulatory center of the hypothalamus. The descending pathways from the hypothalamus via brainstem/spinal cord and the adrenals to the skin are involved in maintaining normal skin temperature. The action of NE and 5-HT reuptake inhibitors is known to impinge on both the CNS and peripheral nervous system (PNS). The pathophysiology of VMS is mediated by both central and peripheral mechanisms and, therefore, the interplay between the CNS and PNS may account for the efficacy of dual acting SRI/NRIs in the treatment of thermoregulatory dysfunction. In fact, the physiological aspects and the CNS/PNS involvement in VMS may account for the lower doses proposed to treat VMS (Loprinzi, et al., Lancet, 2000, 356:2059-2063; Stearns et al., JAMA, 2003, 289:2827-2834) compared to doses used to treat the behavioral aspects of depression. The interplay of the CNS/PNS in the pathophysiology of VMS supports the claims that the norepinephrine system could be targeted to treat VMS.

Although VMS are most commonly treated by hormone therapy, some patients cannot tolerate estrogen treatment (Berendsen, Maturitas, 2000, 36(3): 155-164, Fink et al., Nature, 1996, 383(6598): 306). In addition, hormone replacement therapy is usually not recommended for women or men with or at risk for hormonally sensitive cancers (e.g. breast or prostate cancer). Thus, non-hormonal therapies (e.g. fluoxetine, paroxetine [SRIs] and clonidine) are being evaluated clinically. WO9944601 discloses a method for decreasing hot flushes in a human female by administering fluoxetine. Other options have been studied for the treatment of hot flushes, including steroids, alpha-adrenergic agonists, and beta-blockers, with varying degree of success (Waldinger et al., Maturitas, 2000, 36(3): 165-168).

α₂-Adrenergic receptors play a role in thermoregulatory dysfunctions (Freedman et al., Fertility & Sterility, 2000, 74(1): 20-3). These receptors are located both pre- and post-synaptically and mediate an inhibitory role in the central and peripheral nervous system. There are four distinct subtypes of the adrenergic_{α2} receptors, i.e., are α_{2A}, α_{2B}, α_{2C} and α_{2D} (Mackinnon et al., TIPS, 1994, 15: 119; French, Pharmacol. Ther., 1995, 68: 175). A non-select α₂-adrenoceptor antagonist, yohimbine, induces a flush and an α₂-adrenergic receptor agonist, clonidine, alleviates the yohimbine effect (Katovich, et al., Proceedings of the Society for Experimental Biology & Medicine, 1990, 193(2): 129-35, Freedman et al., Fertility & Sterility, 2000, 74(1): 20-3). Clonidine has been used to treat hot flush. However, using such treatment is associated with a number of undesired side effects caused by high doses necessary to abate hot flush described herein and known in the related arts.

Chronic pain comes in many forms including visceral, inflammatory or neuropathic and crosses all therapeutic areas. It is a debilitating condition that exerts a high social cost in terms of productivity, economic impact and quality of life and current therapies have limited efficacy. Currently, first-line pharmacological treatments for neuropathic pain (i.e., diabetic neuropathy and post-herpetic neuralgia) and fibromyalgia include off-label use of the tricyclic (TCA) antidepressants (e.g., amytriptyline) and anticonvulsants (e.g., gabapentin) (Collins et al., J. Pain Symptom Manage. 2000, 20(6):449-58; and Marcus Expert Opin Pharmacother. 2003, 4(10): 1687-95.). However, these therapies are only effective in 30-50% of patients and produce only a partial reduction in pain (˜50%). In addition, the clinical benefits of these therapies are often outweighed by the side effects, including dry mouth and sedation. Therefore, newer classes of compounds, including non-TCA antidepressants, are being evaluated preclinically and clinically for chronic pain indications, and recently duloxetine was approved for the treatment of diabetic neuropathy. Although more tolerable than the older tricyclic antidepressants, these newer compounds are not devoid of side effects that include sexual dysfunction, weight gain and nausea.

While the precise pathophysiological mechanisms involved in the development and maintenance of chronic pain states are not fully understood, the pathways involved in pain perception and modulation have been well described and characterized (Gebhart, In: Yaksh T L, editor. Spinal afferent processing, New York: Plenum, 1986. pp 391-416; Fields, et al., Annual Review of Neuroscience 1991, 14: 219-245; Fields, et al. In: Wall P D, Melzack R, editors. Textbook of pain, London: Churchill Livingstone, 1999, pp 309-329; Millan, et al. Progress in Neurobiology; 2002, 66:355-474). A major component of this descending pain inhibitory system involves the noradrenergic pathway (Zhuo, et al., Brain Research 1991; 550:35-48; Holden, et al. Neuroscience 1999; 91: 979-990). It is assumed that norepinephrine (NE) and to a lesser extent serotonin (5-HT) reuptake inhibitors (NRIs and SRIs) attenuate pain by preventing presynaptic reuptake of NE/5-HT leading to increased postsynaptic NE/5-HT levels and sustained activation of this descending pain inhibitory pathway. A meta-analysis of antidepressants and neuropathic pain comparing the efficacy of known NRIs, mixed NRI/SRIs and SRIs determined that compounds with NRI activity were more effective in reducing pain, and that select SRIs did not significantly differ from placebo (Collins et al., J. Pain Symptom Manage. 2000, 20(6): 449-58). This analysis suggests that compounds with greater NRI versus SRI activity will be more effective for the treatment of pain.

Given the complex multifaceted nature of pain and of thermoregulation and the interplay between the CNS and PNS in maintaining thermoregulatory the homeostasis, multiple therapies and approaches can be developed to target the treatment of pain and vasomotor symptoms. The present invention provides novel compounds and compositions containing these compounds directed to these and other important uses.

### SUMMARY

The present invention is directed to dihydrobenzfuran derivatives, which are monoamine reuptake inhibitors, compositions containing these derivatives, and methods of their use for the prevention and treatment of conditions, including, inter alia, vasomotor symptoms (such as hot flush), sexual dysfunction (such as desire-related or arousal-related dysfunction), gastrointestinal disorders and genitourinary disorder (such as stress incontinence or urge incontinence), chronic fatigue syndrome, fibromyalgia syndrome, depression disorders (such as major depressive disorder, generalized anxiety disorder, panic disorder, attention deficit disorder with or without hyperactivity, sleep disturbance, and social phobia), diabetic neuropathy, pain, and combinations thereof.

In one embodiment, the present invention is directed to compounds of formula I:

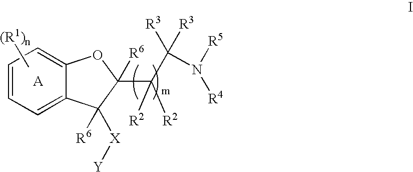

or a pharmaceutically acceptable salt thereof;
wherein:
m is an integer from 0 to 3;
n is an integer from 0 to 4;
X is O, S, SO₂, or NR⁷;
Y is aryl substituted with 0-3 R¹ or heteroaryl substituted with 0-3 R¹;
R¹ is, independently at each occurrence, alkyl, alkoxy, halo, CF₃, OCF₃, arylalkoxy substituted with 0-3 R⁸, hydroxy, alkanoyloxy, nitro, nitrile, alkenyl, alkynyl, alkylsulfoxide, arylsulfoxide substituted with 0-3 R⁸, alkylsulfone, arylsulfone substituted with 0-3 R⁸, alkylsulfonamide, arylsulfonamide substituted with 0-3 R⁸, heteroarylsulfonamide substituted with 0-3 R⁸, heteroarylmethoxy substituted with 0-3 R⁸, alkylamido, or arylamido substituted with 0-3 R³; or
two adjacent R¹ also represent methylenedioxy;
each R² is, independently at each occurrence, H, F, C₁-C₄ alkyl, or OR⁹;
each R³ is, independently at each occurrence, H, F, C₁-C₄ alkyl, or OR¹⁰;
R⁴ is H, C₁-C₄ alkyl, arylalkyl substituted with 0-3 R¹¹, heteroarylalkyl substituted with 0-3 R¹¹, cycloheptylmethyl, cyclohexylmethyl, cyclopentylmethyl, or cyclobutylmethyl; or
one R² and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
one R³ and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
R⁵ is H, C₁-C₄ alkyl, arylalkyl substituted with 0-3 R¹², heteroarylalkyl substituted with 0-3 R¹², cycloheptylmethyl, cyclohexylmethyl, cyclopentylmethyl, or cyclobutylmethyl; or
R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃;
each R⁶ is, independently at each occurrence, H, alkyl, or perfluoroalkyl;
R⁷ is H, alkyl, or aryl substituted with 0-3 R¹³; or
R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃;
R⁸, R¹¹, R¹², and R¹³ are, independently at each occurrence, alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or
two adjacent R⁸, or two adjacent R¹¹, or two adjacent R¹², or two adjacent R¹³ also represent methylenedioxy;
R⁹ and R¹⁰ are, independently at each occurrence, H or C₁-C₄ alkyl; and
wherein
1-3 carbon atoms in ring A may optionally be replaced with N.

In yet other embodiments, the present invention is directed to compositions, comprising:

a. at least one compound of formula I; and
b. at least one pharmaceutically acceptable carrier.

In another embodiment, the invention is directed to methods for treating or preventing a condition selected from the group consisting of a vasomotor symptom, sexual dysfunction, gastrointestinal disorder, genitourinary disorder, chronic fatigue syndrome, fibromyalgia syndrome, depression disorder, endogenous behavioral disorder, cognitive disorder, diabetic neuropathy, pain, and combinations thereof in a subject in need thereof, comprising the step of:

administering to said subject an effective amount of a compound of formula I or pharmaceutically acceptable salt thereof.

In a further embodiment, the invention provides a process for the preparation of a compound of formula Ia

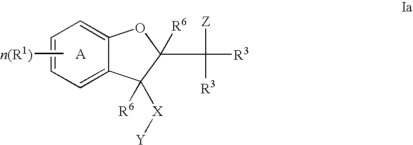

wherein:
z is OH or

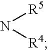

n is an integer from 0 to 4;
X is O, S, SO₂, or NR⁷;
Y is aryl substituted with 0-3 R¹ or heteroaryl substituted with 0-3 R¹;
R¹ is, independently at each occurrence, alkyl, alkoxy, halo, CF₃, OCF₃, arylalkoxy substituted with 0-3 R⁸, hydroxy, alkanoyloxy, nitro, nitrile, alkenyl, alkynyl, alkylsulfoxide, arylsulfoxide substituted with 0-3 R⁸, alkylsulfone, arylsulfone substituted with 0-3 R⁸, alkylsulfonamide, arylsulfonamide substituted with 0-3 R⁸, heteroarylsulfonamide substituted with 0-3 R⁸, heteroarylmethoxy substituted with 0-3 R⁸, alkylamido, or arylamido substituted with 0-3 R³; or
two adjacent R¹ also represent methylenedioxy;
each R³ is, independently at each occurrence, H, F, C₁-C₄ alkyl, or OR¹⁰;
R⁴ is H, C₁-C₄ alkyl, arylalkyl substituted with 0-3 R¹¹, heteroarylalkyl substituted with 0-3 R¹¹, cycloheptylmethyl, cyclohexylmethyl, cyclopentylmethyl, or cyclobutylmethyl; or
one R³ and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
R⁵ is H, C₁-C₄ alkyl, arylalkyl substituted with 0-3 R¹², heteroarylalkyl substituted with 0-3 R¹², cycloheptylmethyl, cyclohexylmethyl, cyclopentylmethyl, or cyclobutylmethyl; or
R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃;
each R⁶ is, independently at each occurrence, H, alkyl, or perfluoroalkyl;
R⁷ is H, alkyl, or aryl substituted with 0-3 R¹³; or
R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃;
R⁸, R¹¹, R¹², and R¹³ are, independently at each occurrence, alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or
two adjacent R⁸, or two adjacent R¹¹, or two adjacent R¹², or two adjacent R¹³ also represent methylenedioxy;
R¹⁰ is H or C₁-C₄ alkyl; and
wherein
1-3 carbon atoms in ring A may optionally be replaced with N,
which process comprises subjecting a compound of formula II below to a ring-closing reaction to form the dihyrobenzofuran ring of formula Ia above

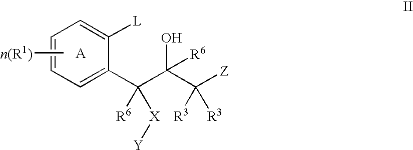

wherein
Z, n, X, Y, R¹, R³, R⁶ and A are as defined hereinabove for formula Ia and wherein L is a leaving group.

### DETAILED DESCRIPTION OF THE INVENTION

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and Δ “ED₅₀ value” means dose which results in 50% alleviation of the observed condition or effect (50% mean maximum endpoint).

“Norepinephrine transporter” is abbreviated NET.

“Human norepinephrine transporter” is abbreviated hNET.

“Serotonin transporter” is abbreviated SERT.

“Human serotonin transporter” is abbreviated hSERT.

“Norepinephrine reuptake inhibitor” is abbreviated NRI.

“Selective norepinephrine reuptake inhibitor” is abbreviated SNRI.

“Serotonin reuptake inhibitor” is abbreviated SRI.

“Selective serotonin reuptake inhibitor” is abbreviated SSRI.

“Norepinephrine” is abbreviated NE.

“Serotonin is abbreviated 5-HT.

“Subcutaneous” is abbreviated sc.

“Intraperitoneal” is abbreviated ip.

“Oral” is abbreviated po.

In the context of this disclosure, a number of terms shall be utilized. The terms “treat,” “treatment” and “treating” as used herein includes preventative (e.g., prophylactic), curative or palliative treatment.

The term “effective amount,” as used herein, refers to an amount effective, at dosages, and for periods of time necessary, to achieve the desired result with respect to treatment of a given disease or disorder. An effective amount is also one in which any toxic or detrimental effects of the components are outweighed by the therapeutically beneficial effects. In particular, with respect to vasomotor symptoms, “effective amount” refers to the amount of compound or composition of compounds that would increase norepinephrine levels to compensate in part or total for the lack of steroid availability in subjects subject afflicted with a vasomotor symptom. Varying hormone levels will influence the amount of compound required in the present invention. For example, the pre-menopausal state may require a lower level of compound due to higher hormone levels than the peri-menopausal state.

The effective amount of components of the present invention will vary from patient to patient not only with the particular compound, component or composition selected, the route of administration, and the ability of the components (alone or in combination with one or more additional active agents) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response.

Preferably, the compounds of the present invention are administered at a dosage and for a time such that the number of hot flushes is reduced as compared to the number of hot flushes prior to the start of treatment. Such treatment can also be beneficial to reduce the overall severity or intensity distribution of any hot flushes still experienced, as compared to the severity of hot flushes prior to the start of the treatment. With respect to sexual dysfunction, gastrointestinal disorder, genitourinary disorder, chronic fatigue syndrome, fibromyalgia syndrome, depression disorder, endogenous behavioral disorder, cognitive disorder, diabetic neuropathy, or pain, the compounds of the present invention are administered at a dosage and for a time sufficient to treat the symptom or condition.

For example, for a patient, compounds of formula I, or a pharmaceutically acceptable salt thereof, may be administered, preferably, at a dosage of from about 0.1 mg/day to about 1500 mg/day, dosed one or two times daily, more preferably from about 1 mg/day to about 200 mg/day and most preferably from about 1 mg/day to 100 mg/day for a time sufficient to reduce and/or substantially eliminate the number and/or severity of hot flushes or symptom or condition of the sexual dysfunction, gastrointestinal disorder, genitourinary disorder, chronic fatigue syndrome, fibromyalgia syndrome, depression disorder, endogenous behavioral disorder, cognitive disorder, diabetic neuropathy, or pain.

The terms “component”, “composition”, “composition of compounds”, “compound”, “drug”, or “pharmacologically active agent” or “active agent” or “medicament” are used interchangeably herein to refer to a compound or compounds or composition of matter which, when administered to a subject (human or animal) induces a desired pharmacological and/or physiologic effect by local and/or systemic action.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

As used herein, the term “inhibitor” refers to any agent that inhibits, suppresses, represses, or decreases a specific activity, such as norepinephrine reuptake activity. The term “inhibitor” is intended to comprise any compound, e.g., antibody, small molecule, peptide, oligopeptide, polypeptide, or protein (preferably small molecule or peptide) that exhibits a partial, complete, competitive and/or inhibitory effect on mammalian, preferably human, norepinephrine reuptake or both serotonin reuptake and the norepinephrine reuptake, thus diminishing or blocking (preferably diminishing) some or all of the biological effects of endogenous norepinephrine reuptake or of both serotonin reuptake and the norepinephrine reuptake.

Within the present invention, the compounds of formula I may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

“Administering,” as used herein, means either directly administering a compound or composition of the present invention, or administering a prodrug, derivative or analog which will form an equivalent amount of the active compound or substance within the body.

The term “subject” or “patient” refers to an animal including the human species that is treatable with the compounds, compositions, and/or methods of the present invention. The term “subject” or “subjects” is intended to refer to both the male and female gender unless one gender is specifically indicated. Accordingly, the term “patient” comprises any mammal which may benefit from treatment of a disease or disorder, such as a human, especially if the mammal is female, either in the pre-menopausal, peri-menopausal, or post-menopausal period. Furthermore, the term patient includes female animals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis. However, the term “patient” is not intended to be limited to a woman.

“Side effect” refers to a consequence other than the one(s) for which an agent or measure is used, such as one or more adverse effects produced by a drug, especially on a tissue or organ system other than the one sought to be benefited by its administration. In the case, for example, of high doses of NRIs or NRI/SRI compounds alone, the term “side effect” may refer to such conditions as, for example, vomiting, nausea, sweating, and flushes (Janowsky, et al., Journal of Clinical Psychiatry, 1984, 45(10 Pt 2): 3-9).

“Vasomotor symptoms,” “vasomotor instability symptoms” and “vasomotor disturbances” include, but are not limited to, hot flushes (flushest), insomnia, sleep disturbances, mood disorders, irritability, excessive perspiration, night sweats, fatigue, and the like, caused by, inter alia, thermoregulatory dysfunction.

The term “hot flush” (also called “hot flash”) is an art-recognized term that refers to an episodic disturbance in body temperature typically consisting of a sudden skin flushing, usually accompanied by perspiration in a subject.

The terms “premature menopause” or “artificial menopause” refer to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

The term “pre-menopausal” means before the menopause, the term “peri-menopausal” means during the menopause and the term “post-menopausal” means after the menopause. “Ovariectomy” means removal of an ovary or ovaries and can be effected according to Merchenthaler et al., Maturitas, 1998, 30(3): 307-316.

The term “sexual dysfunction” includes, but is not limited to, condition relating to defects in sexual desire and/or arousal.

As used herein, “gastrointestinal and genitourinary disorders” includes irritable bowel syndrome, symptomatic GERD, hypersensitive esophagus, nonulcer dyspepsia, noncardiac chest pain, biliary dyskinesia, sphincter of Oddi dysfunction, incontinence (i.e., urge incontinence, stress incontinence, genuine stress incontinence, and mixed incontinence, including the involuntary voiding of feces or urine, and dribbling or leakage or feces or urine which may be due to one or more causes including but not limited to pathology altering sphincter control, loss of cognitive function, overdistention of the bladder, hyperreflexia and/or involuntary urethral relaxation, weakness of the muscles associated with the bladder or neurologic abnormalities), interstitial cystitis (irritable bladder), and chronic pelvic pain (including, but not limited to vulvodynia, prostatodynia, and proctalgia).

As used herein, “chronic fatigue syndrome” (CFS) is a condition characterized by physiological symptoms selected from weakness, muscle aches and pains, excessive sleep, malaise, fever, sore throat, tender lymph nodes, impaired memory and/or mental concentration, insomnia, disordered sleep, localized tenderness, diffuse pain and fatigue, and combinations thereof, whether or not correlated with Epstein-Barr virus infection.

As used herein, “fibromyalgia syndrome” (FMS) includes FMS and other somatoform disorders, including FMS associated with depression, somatization disorder, conversion disorder, pain disorder, hypochondriasis, body dysmorphic disorder, undifferentiated somatoform disorder, and somatoform NOS. FMS and other somatoform disorders are accompanied by physiological symptoms selected from a generalized heightened perception of sensory stimuli, abnormalities in pain perception in the form of allodynia (pain with innocuous stimulation), abnormalities in pain perception in the form of hyperalgesia (increased sensitivity to painful stimuli), and combinations thereof.

As used herein, the term “depression disorder” includes major depressive disorder, generalized anxiety disorder, panic disorder, attention deficit disorder with or without hyperactivity, sleep disturbance, social phobia, and combinations thereof.

The compounds of the present invention can also be used to treat a cognitive disorder or an endogenous behavioral disorder. As used herein, a “cognitive disorder” includes changes or defects in alertness; mild cognitive impairment (MCI), characterized by problems with memory, language, or other mental functions which is severe enough to be noticeable or be detected by tests, but not serious enough to significantly interfere with daily life; cognitive disorder NOS (not otherwise specified), characterized by a syndrome of cognitive impairment that does not meet the criteria for delerium, dementia or amnesic disorders; age-related cognitive decline (ARCD); and cognitive arousal, such as increased arousal states. A cognition disorder can be ideopathic, or can be caused by a variety of other factors such as a congenital defect, alcohol or drug addiction, transient or permanent pharmacologic effects of drugs, organic or infectious disease (e.g., Alzheimer's disease, Parkinson's disease, AIDS), trauma (e.g., brain injury, stroke) or advanced age. As used herein, an “endogenous behavioral disorder” includes attention deficit disorder/attention deficit hyperactivity disorder (ADD/ADHD, including adult and pediatric forms of predominantly inattentive, predominantly hyperactive, or combined types), obsessive-compulsive disorder (OCD), oppositional or oppositional explosive defiant disorder (ODD/OEDD), anxiety and panic disorders (APD) and temper, rage and outburst behavior disorder (TROBD).

As used herein, “pain” includes both acute and chronic nociceptic or neuropathic pain, which may be centralized pain, peripheral pain, or combination thereof. The term includes many different types of pain, including, but not limited to, visceral pain, musculoskeletal pain, bony pain, cancer pain, inflammatory pain, and combinations thereof, such as lower back pain, atypical chest pain, headache such as cluster headache, migraine, herpes neuralgia, phantom limb pain, pelvic pain, myofascial face pain, abdominal pain, neck pain, central pain, dental pain, opioid resistant pain, visceral pain, surgical pain, bone injury pain, pain during labor and delivery, pain resulting from burns, post partum pain, angina pain, peripheral neuropathy and diabetic neuropathy, post-operative pain, and pain which is co-morbid with nervous system disorders described herein.

As used herein, the term “acute pain” refers to centralized or peripheral pain that is intense, localized, sharp, or stinging, and/or dull, aching, diffuse, or burning in nature and that occurs for short periods of time.

As used herein, the term “chronic pain” refers to centralized or peripheral pain that is intense, localized, sharp, or stinging, and/or dull, aching, diffuse, or burning in nature and that occurs for extended periods of time (i.e., persistent and/or regularly reoccurring), including, for the purpose of the present invention, neuropathic pain and cancer pain. Chronic pain includes neuropathic pain, hyperalgesia, and/or allodynia.

As used herein, the term “neuropathic pain” refers to chronic pain caused by damage to or pathological changes in the peripheral or central nervous systems. Examples of pathological changes related to neuropathic pain include prolonged peripheral or central neuronal sensitization, central sensitization related damage to nervous system inhibitory and/or exhibitory functions and abnormal interactions between the parasympathetic and sympathetic nervous systems. A wide range of clinical conditions may be associated with or form the basis for neuropathic pain including, for example, diabetes, post traumatic pain of amputation (nerve damage cause by injury resulting in peripheral and/or central sensitization such as phantom limb pain), lower back pain, cancer, chemical injury, toxins, other major surgeries, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, or viral or bacterial infections such as shingles or human immunodeficiency virus (HIV), and combinations thereof. Also included in the definition of neuropathic pain is a condition secondary to metastatic infiltration, adiposis dolorosa, burns, central pain conditions related to thalamic conditions, and combinations thereof.

As used herein, the term “hyperalgesia” refers to pain where there is an increase in sensitivity to a typically noxious stimulus.

As used herein, the term “allodynia” refers to an increase in sensitivity to a typically non-noxious stimulus.

As used herein, the term “visceral pain” refers to pain associated with or resulting from maladies of the internal organs, such as, for example, ulcerative colitis, irritable bowel syndrome, irritable bladder, Crohn's disease, rheumatologic (arthralgias), tumors, gastritis, pancreatitis, infections of the organs, biliary tract disorders, and combinations thereof.

As used herein, the term “female-specific pain” refers to pain that may be acute and/or chronic pain associated with female conditions. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes, and combinations thereof.

“Alkyl,” as used herein refers to a straight-chain or branched saturated hydrocarbon group. Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl), and the like. A lower alkyl group typically has up to 6 carbon atoms. In various embodiments, an alkyl group has 1-6 carbon atoms, and is referred to as a “C₁₋₆ alkyl group.” Examples of C₁₋₆ alkyl groups include, but are not limited to, methyl, ethyl, propyl (e.g., n-propyl and isopropyl), butyl (e.g., n-butyl, isobutyl, s-butyl, t-butyl), pentyl (e.g., n-pentyl, neopentyl, isopentyl, t-pentyl), and hexyl groups (e.g., n-hexyl, isohexyl). A branched alkyl group has at least 3 carbon atoms (e.g., an isopropyl group), and in various embodiments, has up to 6 carbon atoms, i.e., a branched lower alkyl group. Examples of branched lower alkyl groups include, but are not limited to:

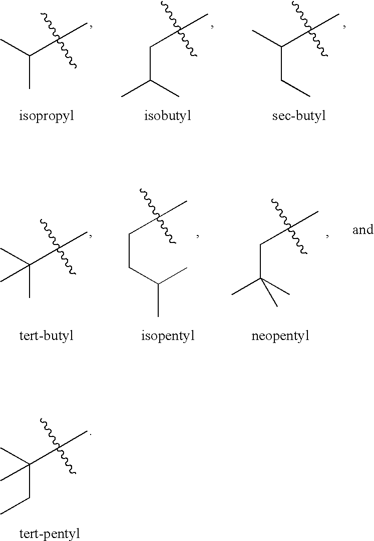

“Alkenyl,” as used herein, refers to an alkyl group of at least two carbon atoms having one or more double bonds, wherein alkyl is as defined herein. Alkenyl groups can be optionally substituted.

“Alkynyl,” as used herein, refers to an alkyl group of at least two carbon atoms having one or more triple bonds, wherein alkyl is as defined herein. Alkynyl groups can be optionally substituted.

“Halo,” as used herein, refers to chloro, bromo, fluoro, and iodo.

“Aryl” as used herein, refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

“Heteroaryl,” as used herein, refers to an optionally substituted, mono-, di-, tri-, or other multicyclic aromatic ring system that includes at least one, and preferably from 1 to about 4 heteroatom ring members selected from sulfur, oxygen and nitrogen. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

“Heterocyclic ring,” as used herein, refers to a stable 4- to 12-membered monocyclic or bicyclic or 7- to 10-membered bicyclic heterocyclic ring that is saturated, partially unsaturated or unsaturated (aromatic), and which contains carbon atoms and from 1 to 4 heteroatoms independently selected from the group consisting of N, O and S and including any bicyclic group in which any of the above defined heterocyclic rings is fused to a benzene ring. The nitrogen and sulfur heteroatoms may optionally be oxidized. The heterocyclic ring may be attached to its pendant group at any heteroatom or carbon atom that results in a stable structure. The heterocyclic rings described herein may be substituted on carbon or on a nitrogen atom if the resulting compound is stable. If specifically noted, a nitrogen atom in the heterocycle may optionally be quaternized. It is preferred that when the total number of S and O atoms in the heterocycle exceeds one, then these heteroatoms are not adjacent to one another. It is preferred that the total number of S and O atoms in the heterocycle is not more than two. Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4H-carbazolyl, α-, β-, or γ-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenoxazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred heterocycles include, but are not limited to, pyridinyl, furanyl, thienyl, pyrrolyl, pyrazolyl, imidazolyl, indolyl, benzimidazolyl, 1H-indazolyl, oxazolidinyl, benzotriazolyl, benzisoxazolyl, oxindolyl, benzoxazolinyl, or isatinyl. Also included are fused ring and spiro compounds containing, for example, the above heterocycles.

“Alkoxy,” as used herein, refers to the group R—O— where R is an alkyl group, as defined herein.

“Arylalkyl,” as used herein, refers to the group R′—R— where R′ is an aryl group, as defined herein, and R is an alkyl group, as defined herein.

“Heteroarylalkyl,” as used herein, refers to the group R″—R— where R″ is a heteroaryl group, as defined herein, and R is an alkyl group, as defined herein.

“Arylalkoxy,” as used herein, refers to the group R′—R—O— where R′ is an aryl group, as defined herein, and R is an alkyl group, as defined herein. An example includes benzyloxy.

“Heteroarylmethoxy,” as used herein, refers to the group R″—CH₂—O— where R″ is a heteroaryl group, as defined herein.

“Alkanoyloxy,” as used herein, refers to the group R—C(═O)—O— where R is an alkyl group, as defined herein, of 1 to 5 carbon atoms.

“Alkylsulfoxide,” as used herein, refers to as used herein, refers to S(═O)—R, where R is alkyl, as defined herein.

“Arylsulfoxide,” as used herein, refers to as used herein, refers to —S(═O)—R′, where R′ is aryl, as defined herein.

“Alkylsulfone,” as used herein, refers to —S(═O)₂—R, where R is alkyl, as defined herein.

“Arylsulfone,” as used herein, refers to —S(═O)₂—R′, where R′ is aryl, as defined herein.

“Alkylsulfonamide,” as used herein, refers to —NR—S(═O)₂—R, where each R is independently, alkyl, as defined above, or the NR part may also be NH.

“Arylsulfonamide,” as used herein, refers to —NR—S(═O)₂—R′, where R is H or alkyl, as defined herein, and R′ is aryl, as defined herein.

“Heteroarylsulfonamide,” as used herein, refers to —NR—S(═O)₂—R″, where R is H or alkyl, as defined herein, and R″ is aryl, as defined herein.

“Alkylamido,” as used herein, refers to —NR—C(═O)—R, where each R is independently, alkyl, as defined above, or the NR part may also be NH.

“Arylamido,” as used herein, refers to —NR—C(═O)—R″, where R is H or alkyl, as defined herein, and R″ is aryl, as defined herein.

“Perfluoroalkyl,” as used herein, refers to an optionally substituted straight or branched aliphatic hydrocarbon chain of 1 to 8 carbon atoms and preferably 1 to 3 carbon atoms, in which all hydrogens are replaced with fluorine.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of another example, the term “5-9 membered heteroaryl group” is specifically intended to individually disclose a heteroaryl group having 5, 6, 7, 8, 9, 5-9, 5-8, 5-7, 5-6, 6-9, 6-8, 6-7, 7-9, 7-8, and 8-9 ring atoms.

In one embodiment, the present invention is directed to compounds of formula I:

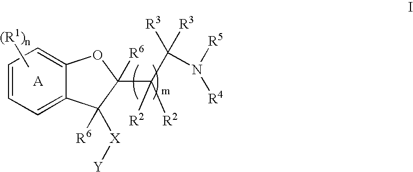

or a pharmaceutically acceptable salt thereof;
wherein:
m is an integer from 0 to 3;
n is an integer from 0 to 4;
X is O, S, SO₂, or NR⁷;
Y is aryl substituted with 0-3 R¹ or heteroaryl substituted with 0-3 R¹;
R¹ is, independently at each occurrence, alkyl, alkoxy, halo, CF₃, OCF₃, arylalkoxy substituted with 0-3 R⁸, hydroxy, alkanoyloxy, nitro, nitrile, alkenyl, alkynyl, alkylsulfoxide, arylsulfoxide substituted with 0-3 R⁸, alkylsulfone, arylsulfone substituted with 0-3 R⁸, alkylsulfonamide, arylsulfonamide substituted with 0-3 R⁸, heteroarylsulfonamide substituted with 0-3 R⁸, heteroarylmethoxy substituted with 0-3 R⁸, alkylamido, or arylamido substituted with 0-3 R³; or
two adjacent R¹ also represent methylenedioxy;
each R² is, independently at each occurrence, H, F, C₁-C₄ alkyl, or OR⁹;
each R³ is, independently at each occurrence, H, F, C₁-C₄ alkyl, or OR¹⁰;
R⁴ is H, C₁-C₄ alkyl, arylalkyl substituted with 0-3 R¹¹, heteroarylalkyl substituted with 0-3 R¹¹, cycloheptylmethyl, cyclohexylmethyl, cyclopentylmethyl, or cyclobutylmethyl; or
one R² and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
one R³ and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
R⁵ is H, C₁-C₄ alkyl, arylalkyl substituted with 0-3 R¹², heteroarylalkyl substituted with 0-3 R¹², cycloheptylmethyl, cyclohexylmethyl, cyclopentylmethyl, or cyclobutylmethyl; or
R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃;
each R⁶ is, independently at each occurrence, H, alkyl, or perfluoroalkyl;
R⁷ is H, alkyl, or aryl substituted with 0-3 R¹³; or
R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃;
R⁸, R¹¹, R¹², and R¹³ are, independently at each occurrence, alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or
two adjacent R⁸, or two adjacent R¹¹, or two adjacent R¹², or two adjacent R¹³ also represent methylenedioxy;
R⁹ and R¹⁰ are, independently at each occurrence, H or C₁-C₄ alkyl; and
wherein
1-3 carbon atoms in ring A may optionally be replaced with N.

In certain preferred embodiments of the compounds of formula I, n is an integer from 0 to 2, and more preferably, 0 to 1.

In certain preferred embodiments of the compounds of formula I, m is an integer from 1 to 2, and more preferably, 1.

In certain preferred embodiments of the compounds of formula I, R¹ is, independently at each occurrence, C₁-C₆ alkyl, alkoxy, halo, CF₃, OCF₃, nitrile, or aryl substituted with 0-3 R⁸. In certain more preferred embodiments, R¹ is, independently at each occurrence, methyl, methoxy, fluoro, chloro, bromo, CF₃, OCF₃, nitrile, or phenyl.

In certain preferred embodiments of the compounds of formula I, R² is, independently at each occurrence, H, fluoro, methyl, ethyl, hydroxy, or methoxy, especially H or hydroxy. In certain preferred embodiments, R² is, independently at each occurrence, hydroxy.

In certain preferred embodiments of the compounds of formula I, R³ is, independently at each occurrence, H, fluoro, methyl, ethyl, or methoxy, especially H at each occurrence.

In certain preferred embodiments of the compounds of formula I, R⁴ is H or C₁-C₄ alkyl, and more preferably, R⁴ is H or methyl.

In certain preferred embodiments of the compounds of formula I, R⁵ is H or C₁-C₄ alkyl and more preferably, R⁵ is H or methyl.

In certain preferred embodiments of the compounds of formula I, one R² and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃.

In certain preferred embodiments of the compounds of formula I, one R³ and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃.

In certain preferred embodiments of the compounds of formula I, R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃. In certain more preferred embodiments of the compounds of formula I, R⁴ and R⁵, together with the nitrogen through which they are attached, form morpholinyl where any carbon ring atom may be optionally substituted with C₁-C₄ alkyl, F, or CF₃.

In certain preferred embodiments of the compounds of formula I, R⁶ is, independently at each occurrence, H, methyl, ethyl, or perfluoromethyl, and more preferably, R⁶ is H at each occurrence.

In certain preferred embodiments of the compounds of formula I, X is NR⁷, especially wherein R⁷ is H, methyl, ethyl or phenyl. In certain other preferred embodiments, X is O. In yet other preferred embodiments, X is S or SO₂.

In certain preferred embodiments of the compounds of formula I, X is NR⁷, especially wherein R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃. In certain even more preferred embodiments, R⁷ and Y, together with the nitrogen through which they are attached, form oxoindolyl, benzimidazolonyl, indolinyl, or indolyl optionally substituted with C₁-C₄ alkyl, F, or CF₃.

Preferred compounds of formula I include:
1-[3-(7-fluoro-3,3-dimethyl-2,3-dihydro-1H-indol-1-yl)-2,3-dihydro-1-benzofuran-2-yl]-N-methylmethanamine;
1-[3-(5-fluoro-3,3-dimethyl-2,3-dihydro-1H-indol-1-yl)-2,3-dihydro-1-benzofuran-2-yl]-N-methylmethanamine;
1-[3-(7-fluoro-3,3-dimethyl-2,3-dihydro-1H-indol-1-yl)-2,3-dihydro-1-benzofuran-2-yl]-N,N-dimethylmethanamine;
1-[3-(7-fluoro-3,3-dimethyl-2,3-dihydro-1H-indol-1-yl)-2,3-dihydro-1-benzofuran-2-yl]methanamine;
1-[3-(7-fluoro-3,3-dimethyl-2,3-dihydro-1H-indol-1-yl)-2,3-dihydro-1-benzofuran-2-yl]-N-methylmethanamine;
N-{[3-(7-fluoro-3,3-dimethyl-2,3-dihydro-1H-indol-1-yl)-2,3-dihydro-1-benzofuran-2-yl]methyl}propan-2-amine;
1-[3-(7-fluoro-3,3-dimethyl-2,3-dihydro-1H-indol-1-yl)-2,3-dihydro-1-benzofuran-2-yl]-N,N-dimethylmethanamine;
N-{[3-(7-fluoro-3,3-dimethyl-2,3-dihydro-1H-indol-1-yl)-2,3-dihydro-1-benzofuran-2-yl]methyl}ethanamine;
1-[3-(2-ethoxyphenoxy)-2,3-dihydro-1-benzofuran-2-yl]-N-methylmethanamine;
1-[3-(2,3-dihydro-1H-indol-1-yl)-2,3-dihydro-1-benzofuran-2-yl]-N-methylmethanamine;
1-[3-(1H-indol-1-yl)-2,3-dihydro-1-benzofuran-2-yl]-N-methylmethanamine;
7-fluoro-1-{7-fluoro-2-[(methylamino)methyl]-2,3-dihydro-1-benzofuran-3-yl}-3,3-dimethyl-1,3-dihydro-2H-indol-2-one;
1-[5-fluoro-3-(7-fluoro-3,3-dimethyl-2,3-dihydro-1H-indol-1-yl)-2,3-dihydro-1-benzofuran-2-yl]-N-methyl methanamine;
1-{6-fluoro-2-[(methylamino)methyl]-2,3-dihydro-1-benzofuran-3-yl}-3,3-dimethyl-1,3-dihydro-2H-indol-2-one;
3,3-dimethyl-1-{2-[(methylamino)methyl]-2,3-dihydro-1-benzofuran-3-yl}-1,3-dihydro-2H-indol-2-one;
1-[3-(3,4-dihydroquinolin-1(2H)-yl)-2,3-dihydro-1-benzofuran-2-yl]-N-methylmethanamine;
N-methyl-2-[(methylamino)methyl]-N-phenyl-2,3-dihydro-1-benzofuran-3-amine;
4-Fluoro-3-{6-fluoro-2-[(methylamino)methyl]-2,3-dihydro-1-benzofuran-3-yl}-1-isopropyl-1,3-dihydro-2H-benzimidazol-2-one;
4-Fluoro-3-{6-fluoro-2-[(methylamino)methyl]-2,3-dihydro-1-benzofuran-3-yl}-1-isopropyl-1,3-dihydro-2H-benzimidazol-2-one;
4-Fluoro-3-{6-fluoro-2-[(methylamino)methyl]-2,3-dihydro-1-benzofuran-3-yl}-1-isopropyl-1,3-dihydro-2H-benzimidazol-2-one and

pharmaceutically acceptable salts thereof.

Particularly preferred compounds of formula I include:
1-[(2R,3S)-3-(7-fluoro-3,3-dimethyl-2,3-dihydro-1H-indol-1-yl)-2,3-dihydro-1-benzofuran-2-yl]-N-methylmethanamine;
1-[(2R,3S)-3-(5-fluoro-3,3-dimethyl-2,3-dihydro-1H-indol-1-yl)-2,3-dihydro-1-benzofuran-2-yl]-N-methylmethanamine;
1-[(2R,3S)-3-(7-fluoro-3,3-dimethyl-2,3-dihydro-1H-indol-1-yl)-2,3-dihydro-1-benzofuran-2-yl]-N,N-dimethylmethanamine;
1-[(2R,3S)-3-(7-fluoro-3,3-dimethyl-2,3-dihydro-1H-indol-1-yl)-2,3-dihydro-1-benzofuran-2-yl]methanamine;
1-[(2S,3R)-3-(7-fluoro-3,3-dimethyl-2,3-dihydro-1H-indol-1-yl)-2,3-dihydro-1-benzofuran-2-yl]-N-methylmethanamine;
N-{[(2S,3R)-3-(7-fluoro-3,3-dimethyl-2,3-dihydro-1H-indol-1-yl)-2,3-dihydro-1-benzofuran-2-yl]methyl}propan-2-amine;
1-[(2S,3R)-3-(7-fluoro-3,3-dimethyl-2,3-dihydro-1H-indol-1-yl)-2,3-dihydro-1-benzofuran-2-yl]-N,N-dimethylmethanamine;
N-{[(2S,3R)-3-(7-fluoro-3,3-dimethyl-2,3-dihydro-1H-indol-1-yl)-2,3-dihydro-1-benzofuran-2-yl]methyl}ethanamine;
1-[(2R,3S)-3-(2-ethoxyphenoxy)-2,3-dihydro-1-benzofuran-2-yl]-N-methylmethanamine;
1-[3-(2,3-dihydro-1H-indol-1-yl)-2,3-dihydro-1-benzofuran-2-yl]-N-methylmethanamine;
1-[3-(1H-indol-1-yl)-2,3-dihydro-1-benzofuran-2-yl]-N-methylmethanamine;
7-fluoro-1-{(2S,3R)-7-fluoro-2-[(methylamino)methyl]-2,3-dihydro-1-benzofuran-3-yl}-3,3-dimethyl-1,3-dihydro-2H-indol-2-one;
1-[(2R,3S)-5-fluoro-3-(7-fluoro-3,3-dimethyl-2,3-dihydro-1H-indol-1-yl)-2,3-dihydro-1-benzofuran-2-yl]-N-methylmethanamine;
1-{6-fluoro-2-[(methylamino)methyl]-2,3-dihydro-1-benzofuran-3-yl}-3,3-dimethyl-1,3-dihydro-2H-indol-2-one;
3,3-dimethyl-1-{2-[(methylamino)methyl]-2,3-dihydro-1-benzofuran-3-yl}-1,3-dihydro-2H-indol-2-one;
1-[3-(3,4-dihydroquinolin-1(2H)-yl)-2,3-dihydro-1-benzofuran-2-yl]-N-methylmethanamine;
N-methyl-2-[(methylamino)methyl]-N-phenyl-2,3-dihydro-1-benzofuran-3-amine;
4-Fluoro-3-{(2SR,3RS)-6-fluoro-2-[(methylamino)methyl]-2,3-dihydro-1-benzofuran-3-yl}-1-isopropyl-1,3-dihydro-2H-benzimidazol-2-one;
4-Fluoro-3-{(2S,3R)-6-fluoro-2-[(methylamino)methyl]-2,3-dihydro-1-benzofuran-3-yl}-1-isopropyl-1,3-dihydro-2H-benzimidazol-2-one;
4-Fluoro-3-{(2R,3S)-6-fluoro-2-[(methylamino)methyl]-2,3-dihydro-1-benzofuran-3-yl}-1-isopropyl-1,3-dihydro-2H-benzimidazol-2-one and

pharmaceutically acceptable salts thereof.

In certain preferred embodiments of the compounds of formula I, the pharmaceutically acceptable salt is a hydrochloride.

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972), the entire disclosures of which are herein incorporated by reference.

The present invention includes prodrugs of the compounds of formula I. “Prodrug,” as used herein, means a compound which is convertible in vivo by chemical or metabolic means (e.g. by hydrolysis) to a compound of formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

Further, the compounds of formula I may exist in unsolvated as well as in solvated forms with pharmaceutically acceptable solvents such as water, ethanol, and the like. In general, the solvated forms are considered equivalent to the unsolvated forms for the purpose of the present invention.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

In one embodiment, the present invention provides a process for the preparation of a compound of formula Ia

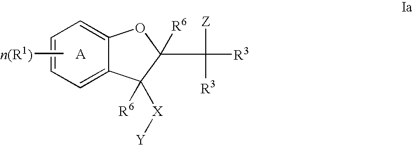

wherein:
z is OH or

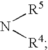

n is an integer from 0 to 4;
X is O, S, SO₂, or NR⁷;
Y is aryl substituted with 0-3 R¹ or heteroaryl substituted with 0-3 R¹;
R¹ is, independently at each occurrence, alkyl, alkoxy, halo, CF₃, OCF₃, arylalkoxy substituted with 0-3 R⁸, hydroxy, alkanoyloxy, nitro, nitrile, alkenyl, alkynyl, alkylsulfoxide, arylsulfoxide substituted with 0-3 R⁸, alkylsulfone, arylsulfone substituted with 0-3 R⁸, alkylsulfonamide, arylsulfonamide substituted with 0-3 R⁸, heteroarylsulfonamide substituted with 0-3 R⁸, heteroarylmethoxy substituted with 0-3 R⁸, alkylamido, or arylamido substituted with 0-3 R³; or
two adjacent R¹ also represent methylenedioxy;
each R³ is, independently at each occurrence, H, F, C₁-C₄ alkyl, or OR¹⁰;
R⁴ is H, C₁-C₄ alkyl, arylalkyl substituted with 0-3 R¹¹, heteroarylalkyl substituted with 0-3 R¹¹, cycloheptylmethyl, cyclohexylmethyl, cyclopentylmethyl, or cyclobutylmethyl; or
R³ and R⁴, together with the nitrogen and carbon through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
R⁵ is H, C₁-C₄ alkyl, arylalkyl substituted with 0-3 R¹², heteroarylalkyl substituted with 0-3 R¹², cycloheptylmethyl, cyclohexylmethyl, cyclopentylmethyl, or cyclobutylmethyl; or
R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃;
each R⁶ is, independently at each occurrence, H, alkyl, or perfluoroalkyl;
R⁷ is H, alkyl, or aryl substituted with 0-3 R¹³; or
R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃;
R⁸, R¹¹, R¹², and R¹³ are, independently at each occurrence, alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or
two adjacent R⁸, or two adjacent R¹¹, or two adjacent R¹², or two adjacent R¹³ also represent methylenedioxy;
R¹⁰ is H or C₁-C₄ alkyl; and
wherein
1-3 carbon atoms in ring A may optionally be replaced with N,
which process comprises subjecting a compound of formula II below to a ring-closing reaction to form the dihyrobenzofuran ring of formula Ia

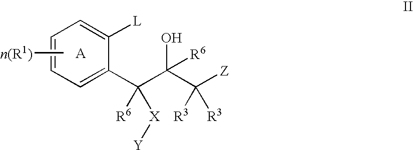

wherein
Z, n, X, Y, R¹, R³, R⁶ and A are the same as defined hereinabove for formula Ia and
wherein
L is a leaving group.

In one embodiment of the process of preparing of a compound of formula Ia, the ring-closing reaction includes an intramolecular nucleophilic replacement of leaving group L of the compound of formula II. In certain embodiments, the intramolecular nucleophilic replacement is performed in a solvent in the presence of a base.

In another embodiment of the process of preparing a compound of formula Ia, the ring-closing reaction is an intramolecular coupling reaction. In certain embodiments, the intramolecular coupling reaction is performed in the presence of a transition metal catalyst and a phosphine ligand. Moreover, in some embodiments, the intramolecular coupling reaction is performed in a solvent in the presence of a base.

In certain preferred embodiments of the process of preparing a compound of formula Ia, Z of the compound of formula II is OH and the process includes converting Z into a leaving group and displacing the leaving group with:

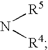

wherein
R⁴ and R⁵ are the same as defined hereinabove for formula Ia.

In other preferred embodiments of the process of preparing a compound of formula Ia, a compound of formula II is formed by opening the epoxide ring of a compound of formula III below

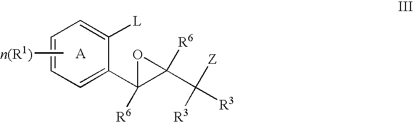

wherein
Z, n, R¹, R³, R⁶ and A are the same as defined herein above for formula Ia and
wherein
L is a leaving group.

In some embodiments of the process, the epoxide ring opening of a compound of formula III includes reacting a suitably nucleophilic compound of formula XY with the epoxide compound, wherein X and Y are the same as defined hereinabove for formula Ia.

In certain embodiments, a compound of formula XY is treated with a base prior to or during its reaction with the epoxide compound of formula Ill. In certain other embodiments, the epoxide compound of formula III is treated with a Lewis acid prior to or during its reaction with a compound of formula XY.

In any of the above-described embodiments, the epoxide ring opening may be performed in the presence of a solvent.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991, the entire disclosure of which is herein incorporated by reference.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

Compounds of the present invention may be suitably prepared in accordance with the following general description. Variables used are as defined for formula I, unless otherwise noted. The reagents used in the preparation of the compounds of this invention can be either commercially obtained or can be prepared by standard procedures described in the literature. In accordance with this invention, compounds of formula I, wherein m of formula I is 0, may be produced by the following reaction schemes (Scheme I and II). Similar reaction schemes may be used to prepare compounds of formula I, wherein m is an integer of 1, 2 or 3.

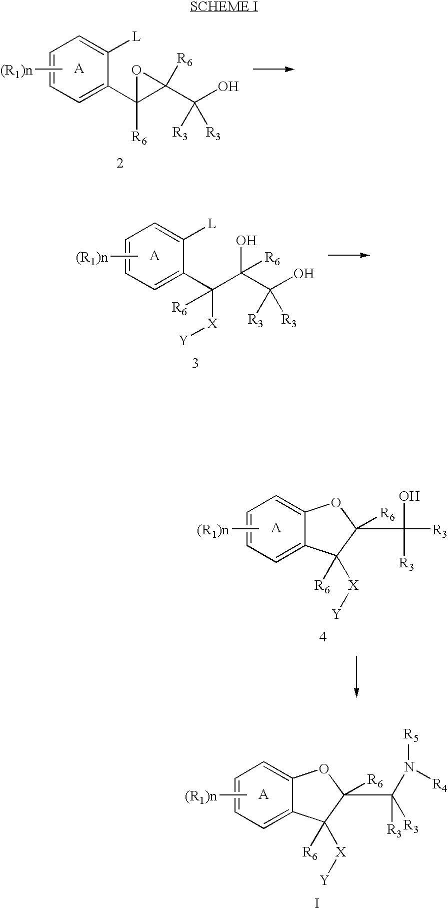

wherein:
X, Y, n, R₁, R₃, R₄, and R₅ are as previously described and
L is a leaving group

As illustrated in Scheme I, compounds of formula 3 may be formed via a regio- and stereo-selective ring opening of an appropriately substituted epoxide of formula 2 (formed via an epoxidation of an appropriately substituted allylic alcohol) with an appropriately substituted compound of formula YX. Any conventional method for the regio- and stereo-selective ring opening of an epoxide can be employed for this conversion. In accordance with the preferred embodiment of this invention, compounds of formula YX are treated with a base, e.g. sodium hydroxide, sodium hydride, sodium tert-butoxide, potassium hydroxide, potassium tert-butoxide, then treated with the epoxide of formula 2 in a suitable solvent including but not limited to THF, DMF, water, methylene chloride, and ethanol. The epoxide of formula 2 can be pre-treated with a Lewis acid, e.g. titanium iso-propoxide, boron-trifluoride, etc. to enhance regio-selective ring opening. The reaction can be effected at the temperature ranging from room temperature to 80° C. over duration of about 2 hours to about 72 hours. Alternatively, compounds of formula YX that are suitably nucleophilic, e.g. indoline, can be heated with the epoxide of formula 2 in the absence of any solvent at temperatures from about 50° C. to about 170° C. to form compounds of formula 3. Furthermore, epoxide ring opening of compounds of formula 2 by compounds of formula YX of suitably nucleophilic, e.g. indolines, can also be effected in the presence of Lewis acid such as titanium iso-propoxide in a suitable solvent, e.g. THF at room temperature. The ring closure to form dihydrobenzofuran 4 can be effected by various reaction executions including but not limited to the intramolecular nucleophilic replacement of leaving group L or intramolecular coupling reaction of compounds of formula 3. The nucleophilic replacement is typically executed by treating the compounds of formula 3 with a suitable base including but not limited to potassium tert-butoxide, sodium hydroxide, or sodium hydride in a suitable solvent including but not limited to DMF and THF at the room temperature to 80° C. The coupling reaction is typically catalyzed by a transition metal in the presence of a suitable phosphine ligand. In accordance with the preferred embodiment of this invention, the coupling reaction can be executed in a suitable solvent such as THF, ether, toluene, and benzene in the presence of a suitable base such as Cs₂CO₃, KF, and K₃PO₄ at the temperature ranging from room temperature to the boiling point of the solvent used under an inert atmosphere of nitrogen or argon. The transition metal catalyst used in the coupling reaction include but not limited to palladium and nickel in the format of such as Pd(OAc)₂ and nickel chloride. A suitable phosphine ligand can include, but is not limited to, 2-dicyclohexylphosphino-2′,4′,6-tri-1-propyl-1,1′-biphenyl, 2-(di-t-butylphosphino) biphenyl, 2-dicyclohexyl phosphino-2′-(m,n-dimethylamino)biphenyl and 2-(di-cyclohexyl phosphino)biphenyl). Conversion of compounds of formula 4 to the compounds of formula I of this invention can be furnished by selectively converting the terminal alcohol into a leaving group and displacing it with a desired amine. Any conventional method for the selective conversion of a terminal alcohol into a leaving group, and any conventional method for displacing a terminal leaving group with an amine can be utilized for this conversion. In accordance with the preferred embodiment of this invention, the alcohol of formula 4 can be treated with para-toluenesulfonyl chloride in pyridine to form the tosylate, which can be converted to the compounds of formula I, the compounds of this invention, by treatment with an excess of alcoholic amine solution, either at room temperature or heated to about 40° C. to about 80° C. in a sealed tube. Alternatively, the terminal hydroxyl group of compounds of formula 4 can be activated via a Mitsunobu protocol and treated with a suitable sulfamide and amide followed by removal of the protection group to afford the desired amines of formula I. The Mitsunobo procedure is well documented (e.g. Hughes, David L. Organic Preparations and Procedures International (1996), 28(2), 127-64.), the entire disclosure of which is herein incorporated by reference. In accordance with the preferred embodiment of this invention, compounds of formula I may be effected by treatment of a mixture of compounds of formula 4, and a suitable 2-nitrobenzene sulfamide and triphenylphosphine in an aprotic solvent such as THF with DIAD (diisopropyl azodicarboxylate). The reaction is generally executed at the room temperature under a blanket of inert gas with an approximate duration of 2 to 72 hours. The 2-nitro benzene sulfonyl group can be subsequently removed by treatment of Mitsunobo adduct with a soft nucleophiles such as thiophenol to generate compounds of formula I. The free base of compounds of formula I can be converted to a pharmaceutically acceptable salt using any conventional method.

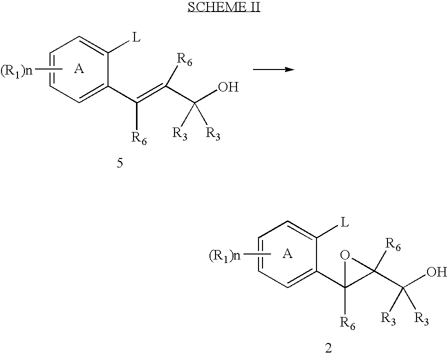

Epoxidation of trans-allylic alcohols can be effected either racemically or asymmetrically using methods described in the literature. In accordance with the preferred embodiment of this invention, racemic epoxidation is conducted with either peracetic acid or meta-chloroperbenzoic acid. If it is desired to produce a single enantiomer of compounds of formula I, asymmetric epoxidation of an allylic alcohol can be performed with tert-butylhydroperoxide or cumene hydroperoxide in the presence of the appropriate tartrate ester, titanium (IV) isopropoxide, and molecular sieves. This method is well established in the literature (e.g., K. B. Sharpless, et. al., J. Org. Chem. 1986, 51, 3710), the entire disclosure of which is herein incorporated by reference. Compounds of formula YX and the starting allylic alcohols are either available from commercial sources or are accessible through methods well established in the literature.

In other embodiments, the invention is directed to pharmaceutical compositions, comprising:

a. at least one compound of formula I, or pharmaceutically acceptable salt thereof; and
b. at least one pharmaceutically acceptable carrier.

Generally, the compound of formula I, or a pharmaceutically acceptable salt thereof, will be present at a level of from about 0.1%, by weight, to about 90% by weight, based on the total weight of the pharmaceutical composition, based on the total weight of the pharmaceutical composition. Preferably, the compound of formula I, or a pharmaceutically acceptable salt thereof, will be present at a level of at least about 1%, by weight, based on the total weight of the pharmaceutical composition. More preferably, the compound of formula I, or a pharmaceutically acceptable salt thereof, will be present at a level of at least about 5%, by weight, based on the total weight of the pharmaceutical composition. Even more preferably, the compound of formula I or a pharmaceutically acceptable salt thereof will be present at a level of at least about 10%, by weight, based on the total weight of the pharmaceutical composition. Yet even more preferably, the compound of formula I, or a pharmaceutically acceptable salt thereof, will be present at a level of at least about 25%, by weight, based on the total weight of the pharmaceutical composition.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985), the entire disclosure of which is herein incorporated by reference. Pharmaceutically acceptable carriers are those that are compatible with the other ingredients in the formulation and biologically acceptable.

The compounds of this invention may be administered enterally (e.g., orally) or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to about 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration.

Liquid pharmaceutical compositions for parenteral administration, which are sterile solutions or suspensions, can be administered by, for example, intramuscular, intraperitoneal or subcutaneous injection. Sterile solutions can also be administered intravenously. Compositions for oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

In another embodiment of the present invention, the compounds useful in the present invention may be administered to a mammal with one or more other pharmaceutically active agents such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof.

The one or more other pharmaceutically active agents may be administered in a therapeutically effective amount simultaneously (such as individually at the same time, or together in a pharmaceutical composition), and/or successively with one or more compounds of the present invention.

The term “combination therapy” refers to the administration of two or more therapeutic agents or compounds to treat a therapeutic condition or disorder described in the present disclosure, for example hot flush, sweating, thermoregulatory-related condition or disorder, or other condition or disorder. Such administration includes use of each type of therapeutic agent in a concurrent manner. In either case, the treatment regimen will provide beneficial effects of the drug combination in treating the conditions or disorders described herein.

The route of administration may be any enteral or parenteral route, which effectively transports the active compound of formula I, or a pharmaceutically acceptable salt thereof, to the appropriate or desired site of action, such as oral, nasal, pulmonary, transdermal, such as passive or iontophoretic delivery, or parenteral, e.g. rectal, depot, subcutaneous, intravenous, intraurethral, intraarticular, intramuscular, intranasal, ophthalmic solution or an ointment. Furthermore, the administration of compound of formula I, or pharmaceutically acceptable salt thereof, with other active ingredients may be consecutive or simultaneous.

In one embodiment, the present invention is directed to methods for treating or preventing a condition selected from the group consisting of a vasomotor symptom, sexual dysfunction, gastrointestinal disorder, genitourinary disorder, chronic fatigue syndrome, fibromyalgia syndrome, depression disorder, endogenous behavioral disorder, cognitive disorder, diabetic neuropathy, pain, and combinations thereof in a subject in need thereof, comprising the step of:

administering to said subject an effective amount of a compound of formula I or pharmaceutically acceptable salt thereof.

In certain embodiments, the vasomotor symptom is hot flush.

In certain embodiments, the sexual dysfunction is desire-related or arousal-related.

In certain embodiments, the gastrointestinal disorder or the genitourinary disorder is stress incontinence or urge incontinence.

In certain embodiments, the condition is chronic fatigue syndrome.

In certain embodiments, the condition is fibromyalgia syndrome.

In certain embodiments, the condition is a depression disorder selected from the group consisting of major depressive disorder, generalized anxiety disorder, panic disorder, attention deficit disorder with or without hyperactivity, sleep disturbance, social phobia, and combinations thereof.

In certain embodiments, the condition is diabetic neuropathy.

In certain embodiments, the condition is pain.

In certain embodiments, the pain is acute centralized pain, acute peripheral pain, or a combination thereof.

In certain embodiments, the pain is chronic centralized pain, chronic peripheral pain, or a combination thereof.

In certain embodiments, the pain is neuropathic pain, visceral pain, musculoskeletal pain, bony pain, cancer pain, inflammatory pain, or a combination thereof.

In certain embodiments, the neuropathic pain is associated with diabetes, post traumatic pain of amputation, lower back pain, cancer, chemical injury, toxins, major surgery, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, viral infection, bacterial infection, metastatic infiltration, adiposis dolorosa, burns, central pain conditions related to thalamic conditions, or a combination thereof.

In certain embodiments, the neuropathic pain is post-herpetic neuralgia.

In certain embodiments, the visceral pain is associated with ulcerative colitis, irritable bowel syndrome, irritable bladder, Crohn's disease, rheumatologic (arthralgias), tumors, gastritis, pancreatitis, infections of the organs, biliary tract disorders, or a combination thereof.

In certain embodiments, the pain is female-specific pain.

The present invention provides a treatment for vasomotor symptoms by methods of recovering the reduced activity of norepinephrine. Without wishing to be bound by any theory, norepinephrine activity in the hypothalamus or in the brainstem can be elevated by (i) blocking the activity of the NE transporter, (ii) blocking the activity of the presynaptic adrenergic_{α2} receptor with an antagonist, or (iii) blocking the activity of 5-HT on NE neurons with a 5-HT₂ₐ antagonist.

The compounds of the invention are also useful to prevent and treat pain. The pain may be, for example, acute pain or chronic pain. The pain may also be centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain or dental pain, or headaches such as migraines or tension headaches, or combinations of these pains. One skilled in the art will recognize that these pains may overlap one another. For example, a pain caused by inflammation may also be visceral or musculoskeletal in nature.

In a preferred embodiment of the present invention the compounds useful in the present invention are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromyalgia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In a more preferred embodiment, the compounds useful in this invention are used to treat chronic pain that is neuropathic pain, visceral pain, musculoskeletal pain, bony pain, cancer pain or inflammatory pain or combinations thereof, in accordance with the methods described herein. Inflammatory pain can be associated with a variety of medical conditions such as osteoarthritis, rheumatoid arthritis, surgery, or injury. Neuropathic pain may be associated with for example diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. The methods of use for compounds of this invention further include treatments in which the neuropathic pain is a condition secondary to metastatic infiltration, adiposis dolorosa, burns, or central pain conditions related to thalamic conditions.

As mentioned previously, the methods of the present invention may be used to treat pain that is somatic and/or visceral in nature. For example, somatic pain that can be treated in accordance with the methods of the present invention include pains associated with structural or soft tissue injury experienced during surgery, dental procedures, burns, or traumatic body injuries. Examples of visceral pain that can be treated in accordance with the methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to the methods of the present invention may also be related to conditions of hyperalgesia, allodynia, or both. Additionally, the chronic pain may be with or without peripheral or central sensitization.

The compounds useful in this invention may also be used to treat acute and/or chronic pain associated with female conditions, which may also be referred to as female-specific pain. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

### 1-[(2R,3S)-3-(7-fluoro-3,3-dimethyl-2,3-dihydro-1H-indol-1-yl)-2,3-dihydro-1-benzofuran-2-yl]-N-methylmethanamine hydrochloride

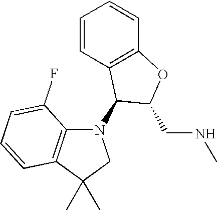

Step 1: A mixture of 2-fluorophenylhydrazine hydrochloride (25 g, 153.8 mmol) and trifluoroacetic acid (34.3 mL, 461.4 mmol) in toluene (770 mL) and acetonitrile (77 mL) was degassed with nitrogen for 20 minutes prior to the addition of a degassed solution of isobutyraldehyde (14 mL, 153.8 mmol). The reaction mixture was heated to 35° C. for 12 hours. Upon disappearance of the hydrazine, the reaction was cooled to 0° C. and sodium borohydride (7 g, 184.6 mmol) added carefully. After completion, toluene was removed in vacuo and a 10% aqueous solution of potassium carbonate (500 mL) was added. The mixture was extracted with ethyl acetate (3×100 mL). The organic layers were combined, dried over sodium sulfate, concentrated, and purified via silica gel column (ISCO, 0-100% ethyl acetate/hexane) to give 7-fluoro-3,3-dimethylindoline as an off-white solid. MS (ES) m/z 166.1

Step 2: A mixture of 2-bromocinnamic acid (50 g, 220.2 mmol), cesium carbonate (107.6 g, 330.3 mol), and methyl iodide (219.8 mL, 3523.2 mmol) in acetone (500 mL) was heated in a sealed vessel for 12 hours. The reaction mixture was filtered and the acetone removed in vacuo. A portion (20 g) of the residue (52.8 g) was taken up in dichloromethane (50 mL) and treated at −78 to 25° C. with neat diisobutylaluminum hydride (18 mL, 103.8 mmol). After 2 hours, the reaction mixture was cooled to −78° C. and carefully quenched with methanol (50 mL). After the mixture was warmed to room temperature, dichloromethane was removed in vacuo and the residue purified via silica gel column (ISCO, 0-70% ethyl acetate/hexane) to give (2E)-3-(2-bromophenyl)prop-2-en-1-ol (12.8 g, 72%). (ES) m/z 211.1.

An oven-dried, 3-neck, 1-L round bottom flask fitted with two oven-dried addition funnels and a rubber septum was charged with diisopropyl D-tartrate (4.4 mL, 21 mmol), 4 Å powdered, activated molecular sieves (˜10 g) and dry dichloromethane (350 mL) under nitrogen. After being cooled to −25° C., to the reaction mixture was added titanium isopropoxide (99.99%, 4.2 mL, 13.98 mmol) slowly via a hypodermic syringe. After being stirred for 10 minutes, anhydrous t-butyl hydroperoxide (˜5.5 M in decane, Fluka, 50.0 mL, 275 mmol, further dried over activated 4 Å molecular sieves pellets 10 min prior to use) was added at a moderate rate via an addition funnel. The resulting mixture was stirred at −20° C. for 30 minutes. (2E)-3-(2-bromophenyl)prop-2-en-1-ol (14.9 g, 69.9 mmol) in dry dichloromethane (20 mL) was added dropwise via an addition funnel while maintaining the temperature at −20° C. After the addition, the reaction mixture was stirred at −20° C. for 1 hour and at −15° C. for another 3 hours. After the reaction was complete, cooled 30% aqueous sodium hydroxide solution (8 mL) saturated with sodium chloride and was added slowly at −20° C. After diethyl ether (100 mL) was added, the cold bath was removed and the mixture was allowed to warm to ˜5° C. and stirred for 1 hour. Magnesium sulfate (anhydrous, ˜20 g) was added and the mixture was stirred for 20 minutes, then filtered through a pad of silica gel, washing with ether (100 mL). The filtrate was concentrated and toluene was used to azeotropically remove excess t-BuOOH. The residual oil was purified on silica gel (ISCO 0-30% ethyl acetate/hexane) to give 3-(2-bromophenyl)oxiran-2-yl]methanol. MS (ES) m/z 228.8. The racemic product was resolved using chiral HPLC (Chiralpak AD-H, 20% MeOH on supercritical fluid 80% CO₂) to yield both [(2R,3R)-3-(2-bromophenyl)oxiran-2-yl]methanol (4.6 g, 29%) and [(2S,3S)-3-(2-bromophenyl)oxiran-2-yl]methanol (2.1 g, 13%).

Step 3: To a mixture of [(2R,3R)-3-(2-bromophenyl)oxiran-2-yl]methanol (2.7 g, 11.8 mmol) and titanium isopropoxide (3.9 mL, 13 mmol) in dichloromethane (40 mL) was added 7-fluoro-3,3-dimethylindoline (1.9 g, 11.8 mmol) in dichloromethane (20 mL). The reaction mixture was stirred for 12 hours and poured into a saturated aqueous solution of ammonium chloride (25 mL) and extracted with ethyl acetate (3×25 mL). The organic layers were combined, dried over sodium sulfate, concentrated, and purified via silica gel column (ISCO, 0-100% ethyl acetate/hexane) to give (2S,3S)-3-(2-bromophenyl)-3-(7-fluoro-3,3-dimethyl-2,3-dihydro-1H-indol-1-yl)propane-1,2-diol as a light pink solid (2.5 g, 56%). MS (ES) m/z 393.7; HRMS: calculated for C₁₉H₂₁BrFNO₂+H⁺, 394.08124; found (ESI, [M+H]⁺), 394.0825.

Step 4: A mixture of (2S,3S)-3-(2-bromophenyl)-3-(7-fluoro-3,3-dimethyl-2,3-dihydro-1H-indol-1-yl)propane-1,2-diol (2.5 g, 5.6 mmol), 2-(di-t-butylphosphino)biphenyl (0.18 g, 0.6 mmol), and cesium carbonate (3 g, 9.2 mmol) in toluene (60 mL) was purged with nitrogen while it was heated to 50° C. Palladium acetate (0.09 g, 0.4 mmol) was added and the reaction mixture heated to 80° C. After kept at 80° C. for 12 hours, the reaction mixture was poured into a solution of saturated aqueous ammonium chloride (50 mL) and extracted with ethyl acetate (2×50 mL). The organic layers were combined, dried over sodium sulfate, concentrated, purified on silica gel column (ISCO 0-100% ethyl acetate/hexane) to give 1-[(2R,3S)-3-(7-fluoro-3,3-dimethyl-2,3-dihydro-1H-indol-1-yl)-2,3-dihydro-1-benzofuran-2-yl]methanol (0.85 g, 43%) which was used in next step without further purification. MS (ES) m/z 314.1.

Step 5: To a solution of 1-[(2R,3S)-3-(7-fluoro-3,3-dimethyl-2,3-dihydro-1H-indol-1-yl)-2,3-dihydro-1-benzofuran-2-yl]methanol (0.65 g, 2.1 mmol), N-methyl-2-nitro-benzenesulfonamide (0.5 g, 2.3 mmol), and triphenylphosphine (1.21 g, 4.6 mmol) in anhydrous tetrahydrofuran (20 mL) was added diisopropylazodicarboxylate (0.89 mL, 4.6 mmol). After stirred at room temperature for 12 hours, the reaction mixture was poured into a solution of saturated ammonium chloride (50 mL) and extracted with ethyl acetate (2×50 mL). The organic layers were combined, dried over sodium sulfate, concentrated, and purified on silica gel column (ISCO 0-100% ethyl acetate/hexane) to give N-[(2R,3S)-3-(7-fluoro-3,3-dimethyl-2,3-dihydro-indol-1-yl)-2,3-dihydro-benzofuran-2-ylmethyl]-N-methyl-2-nitro-benzenesulfonamide (>90%).

Step 6: To a mixture of N-[(2R,3S)-3-(7-fluoro-3,3-dimethyl-2,3-dihydro-indol-1-yl)-2,3-dihydro-benzofuran-2-ylmethyl]-N-methyl-2-nitro-benzene sulfonamide (1.1 g, 2.2 mmol) and potassium carbonate (0.91 g, 6.6 mmol) in dimethylformamide (20 mL) was added thiophenol (0.24 mL, 2.31 mmol). After stirred for 1 hour, the reaction mixture was poured into a solution of saturated ammonium chloride (50 mL) and extracted with ethyl acetate (2×50 mL). The organic layers were combined, dried over sodium sulfate, concentrated, and purified on silica gel column (ISCO 0-30% methanol/methylene chloride) to give 1-[(2R,3S)-3-(7-fluoro-3,3-dimethyl-2,3-dihydro-1H-indol-1-yl)-2,3-dihydro-1-benzofuran-2-yl]-N-methylmethanamine (0.48 g, 61%) as an oil. The hydrochloride salt was prepared by dissolving the free base in ether, cooled to 0° C., and adding 1 N hydrochloric acid in ether. After removal of ether, the residue was triturated with ethyl acetate to afford the title compound as hydrochloride salt as a white solid. MS (ES) m/z 327.0; [α]_{D}²⁵=+143.2° (c=10 mg/mL, MeOH).

### Example 2

### 1-[(2R,3S)-3-(5-fluoro-3,3-dimethyl-2,3-dihydro-1H-indol-1-yl)-2,3-dihydro-1-benzofuran-2-yl]-N-methylmethanamine hydrochloride

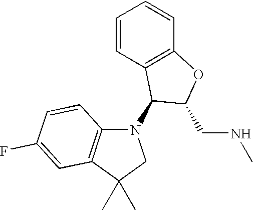

Step 1: In an analogous manner to Example 1, Step 1, 5-fluoro-3,3-dimethylindoline (0.91 g, 57%) was prepared from 4-fluorophenylhydrazine hydrochloride (1.6 g, 10.0 mmol). MS (ES) m/z 166.1.

Step 2: In an analogous manner to Example 1, Step 3, (2S,3S)-3-(2-bromophenyl)-3-(5-fluoro-3,3-dimethyl-2,3-dihydro-1H-indol-1-yl)propane-1,2-diol was prepared from 0.45 g (2.0 mmol) of [(2R,3R)-3-(2-bromophenyl)oxiran-2-yl]methanol and 0.36 (2.0 mmol) g of 5-fluoro-3,3-dimethylindoline. MS (ES) m/z 393.7;

Step 3: In an analogous manner to Example 1, step 4 1-[(2R,3S)-3-(5-fluoro-3,3-dimethyl-2,3-dihydro-1H-indol-1-yl)-2,3-dihydro-1-benzofuran-2-yl]methanol was prepared from (2S,3S)-3-(2-bromophenyl)-3-(5-fluoro-3,3-dimethyl-2,3-dihydro-1H-indol-1-yl)propane-1,2-diol. MS (ES) m/z 314.1.

Step 4: In an analogous manner to Example 1, step 5 N-[(2R,3S)-3-(5-fluoro-3,3-dimethyl-2,3-dihydro-indol-1-yl)-2,3-dihydro-benzofuran-2-ylmethyl]-N-methyl-2-nitro-benzenesulfonamide was prepared from 1-[(2R,3S)-3-(5-fluoro-3,3-dimethyl-2,3-dihydro-1H-indol-1-yl)-2,3-dihydro-1-benzofuran-2-yl]methanol.

Step 5: In an analogous manner to Example 1, step 6, 1-[(2R,3S)-3-(5-fluoro-3,3-dimethyl-2,3-dihydro-1H-indol-1-yl)-2,3-dihydro-1-benzofuran-2-yl]-N-methylmethanamine was prepared from N-[(2R,3S)-3-(5-fluoro-3,3-dimethyl-2,3-dihydro-indol-1-yl)-2,3-dihydro-benzofuran-2-ylmethyl]-N-methyl-2-nitro-benzenesulfonamide.

MS (ES) m/z 326.9; HPLC purity 100.0% at 210-370 nm, 8.5 min.; Xterra RP18, 3.5u, 150×4.6 mm column, 1.2 mL/min, 85/15-5/95 (Ammon. Form. Buff. PH=3.5/CAN+MeOH) for 10 min, hold 4 min.

HRMS: calculated for C₂₀H₂₃FN₂O+H+, 327.18672; found (ESI, [M+H]+), 327.1877

### Example 3

### 1-[(2R,3S)-3-(7-fluoro-3,3-dimethyl-2,3-dihydro-1H-indol-1-yl)-2,3-dihydro-1-benzofuran-2-yl]-N,N-dimethylmethanamine hydrochloride

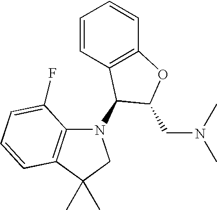

To a solution of 1-[(2R,3S)-3-(7-fluoro-3,3-dimethyl-2,3-dihydro-1H-indol-1-yl)-2,3-dihydro-1-benzofuran-2-yl]methanol (0.08 g, 0.26 mmol) in pyridine (3 mL) was added p-toluenesulfonyl chloride (0.15 g, 0.78 mmol). After stirred at room temperature for 12 hours, the reaction mixture was poured into an aqueous solution of 1 N hydrochloric acid (10 mL) and extracted with ethyl acetate (3×20 mL). The organic layers were combined, washed with saturated aqueous sodium bicarbonate (20 mL), dried over sodium sulfate and concentrated. The crude tosylate residue was taken up in a solution of 33% isopropyl amine in ethanol (10 mL) and heated in a sealed tube at 80° C. for 12 hours. The mixture was cooled to room temperature, poured into a solution of saturated aqueous sodium bicarbonate (10 mL) and extracted with ethyl acetate (3×20 mL). The organic layers were combined, dried over sodium sulfate, concentrated, and purified on silica gel column (ISCO, 40% ethyl acetate/hexane then 10% methanol/dichloromethane) to give 1-[(2R,3S)-3-(7-fluoro-3,3-dimethyl-2,3-dihydro-1H-indol-1-yl)-2,3-dihydro-1-benzofuran-2-yl]-N,N-dimethylmethanamine as an oil. The hydrochloride salt was prepared by dissolving the free base product in ether, cooled to 0° C., and added 1 N hydrochloric acid in ether. After removal of ether, the residue was triturated with ethyl acetate to afford the title compound as hydrochloride salt as a white solid. MS (ES) m/z 341.0; HRMS: calculated for C₂₁H₂₅FN₂O+H+, 341.20237; found (ESI, [M+H]⁺), 341.2022.

### Example 4

### 1-[(2R,3S)-3-(7-fluoro-3,3-dimethyl-2,3-dihydro-1H-indol-1-yl)-2,3-dihydro-1-benzofuran-2-yl]methanamine hydrochloride

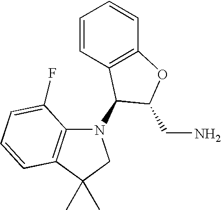

In an analogous manner to Example 3, 1-[(2R,3S)-3-(7-fluoro-3,3-dimethyl-2,3-dihydro-1H-indol-1-yl)-2,3-dihydro-1-benzofuran-2-yl]methanamine hydrochloride was prepared from 1-[(2R,3S)-3-(7-fluoro-3,3-dimethyl-2,3-dihydro-1H-indol-1-yl)-2,3-dihydro-1-benzofuran-2-yl]methanol and 7 N ammonia in ethanol. MS (ES) m/z 312.7.

### Example 5

### 1-[(2S,3R)-3-(7-fluoro-3,3-dimethyl-2,3-dihydro-1H-indol-1-yl)-2,3-dihydro-1-benzofuran-2-yl]-N-methylmethanamine hydrochloride

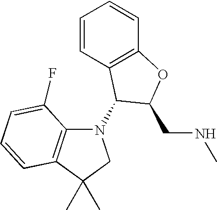

In an analogous manner to Example 3, 1-[(2S,3R)-3-(7-fluoro-3,3-dimethyl-2,3-dihydro-1H-indol-1-yl)-2,3-dihydro-1-benzofuran-2-yl]-N-methylmethanamine hydrochloride was prepared from 1-[(2S,3R)-3-(7-fluoro-3,3-dimethyl-2,3-dihydro-1H-indol-1-yl)-2,3-dihydro-1-benzofuran-2-yl]methanol and 33% wt methylamine in ethanol. HRMS: calculated for C₂₀H₂₃FN₂O+H⁺, 327.18672; found (ESI, [M+H]⁺), 327.1867.

### Example 6

### N-{[(2S,3R)-3-(7-fluoro-3,3-dimethyl-2,3-dihydro-1H-indol-1-yl)-2,3-dihydro-1-benzofuran-2-yl]methyl}propan-2-amine hydrochloride

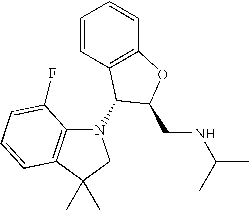

In an analogous manner to Example 3, N-{[(2S,3R)-3-(7-fluoro-3,3-dimethyl-2,3-dihydro-1H-indol-1-yl)-2,3-dihydro-1-benzofuran-2-yl]methyl}propan-2-amine hydrochloride was prepared from 1-[(2S,3R)-3-(7-fluoro-3,3-dimethyl-2,3-dihydro-1H-indol-1-yl)-2,3-dihydro-1-benzofuran-2-yl]methanol and isopropyl amine. MS (ES) m/z 355.1.

### Example 7

### 1-[(2S,3R)-3-(7-fluoro-3,3-dimethyl-2,3-dihydro-1H-indol-1-yl)-2,3-dihydro-1-benzofuran-2-yl]-N,N-dimethylmethanamine hydrochloride

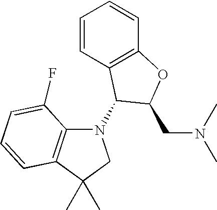

In an analogous manner to Example 3, 1-[(2S,3R)-3-(7-fluoro-3,3-dimethyl-2,3-dihydro-1H-indol-1-yl)-2,3-dihydro-1-benzofuran-2-yl]-N N-dimethylmethanamine hydrochloride was prepared from 1-[(2S,3R)-3-(7-fluoro-3,3-dimethyl-2,3-dihydro-1H-indol-1-yl)-2,3-dihydro-1-benzofuran-2-yl]methanol and dimethyl amine. MS (ES) m/z 341.1; HRMS: calculated for C₂₁H₂₅FN₂O+H⁺, 341.20237; found (ESI, [M+H]⁺), 341.2024.

### Example 8

### N-{[(2S,3R)-3-(7-fluoro-3,3-dimethyl-2,3-dihydro-1H-indol-1-yl)-2,3-dihydro-1-benzofuran-2-yl]methyl}ethanamine hydrochloride

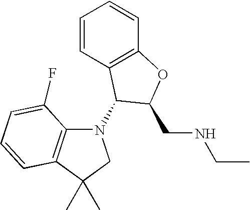

In an analogous manner to Example 3, N-{[(2S,3R)-3-(7-fluoro-3,3-dimethyl-2,3-dihydro-1H-indol-1-yl)-2,3-dihydro-1-benzofuran-2-yl]methyl}ethanamine hydrochloride was prepared from 1-[(2S,3R)-3-(7-fluoro-3,3-dimethyl-2,3-dihydro-1H-indol-1-yl)-2,3-dihydro-1-benzofuran-2-yl]methanol and ethyl amine. MS (ESI) m/z 341.

### Example 9

### 1-[(2R,3S)-3-(2-ethoxyphenoxy)-2,3-dihydro-1-benzofuran-2-yl]-N-methylmethanamine hydrochloride

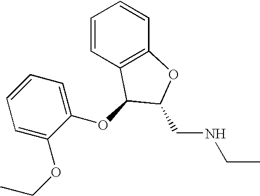

Step 1: A mixture of 2-ethoxyphenol (10 mL, 78 mmol), [(2R,3R)-3-(2-bromophenyl)oxiran-2-yl]methanol (5.95 g, 26 mmol), and sodium hydroxide (1.04 g, 26 mmol) in water (30 mL) were heated to 70° C. for 6 hours. Upon completion, the reaction was poured into saturated aqueous sodium chloride (50 mL) and separated with ether (50 mL). The organic layers were combined and dried with sodium sulfate and the residue purified via silica gel (ISCO 0-100% ethyl acetate/hexane) to give (2R,3S)-3-(2-bromophenyl)-3-(2-ethoxyphenoxy)propane-1,2-diol (6.01 g, 63%). MS (ES) m/z 348.8; HRMS: calculated for C₁₇H₁₉BrO₄+Na+, 389.03589; found (ESI, [M+Na]⁺), 389.0359;

Step 2: In an analogous manner to Example 1, step 4, [(2R,3S)-3-(2-ethoxyphenoxy)-2,3-dihydro-1-benzofuran-2-yl]methanol was prepared from (2R,3S)-3-(2-bromophenyl)-3-(2-ethoxyphenoxy)propane-1,2-diol. MS (ES) m/z 297.2

Step 3: In an analogous manner to Example 1 steps 5 and 6, 1-[(2R,3S)-3-(2-ethoxyphenoxy)-2,3-dihydro-1-benzofuran-2-yl]-N-methylmethanamine hydrochloride as a white solid was prepared from [(2R,3S)-3-(2-ethoxyphenoxy)-2,3-dihydro-1-benzofuran-2-yl]methanol. MS (ES) m/z 300.0; HRMS: calculated for C₁₈H₂₁NO₃+H+, 300.15942; found (ESI, [M+H]⁺), 300.1596.

### Example 10

### (+/−)-1-[3-(2,3-dihydro-1H-indol-1-yl)-2,3-dihydro-1-benzofuran-2-yl]-N-methylmethanamine hydrochloride

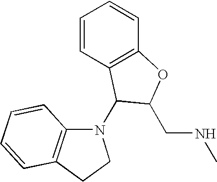

Step 1: A mixture of (2E)-3-(2-bromophenyl)prop-2-en-1-ol (6.4 g, 30 mmol), peracetic acid (15.2 mL, 72 mmol), sodium carbonate (9 g, 84 mmol) in methylene chloride (75 mL) was stirred at room temperature for 18 hours. The mixture was treated with a saturated sodium carbonate solution (50 mL). The organic layer was separated and aqueous layer was extracted with methylene chloride (2×30 mL). The combined organic layers were washed with brine (30 mL), dried (sodium carbonate), and concentrated to give 3-(2-bromophenyl)oxiran-2-yl]methanol as a white solid (5.2 g, 75%). MS (ES) m/z 229.0.

Steps 2-5: In an analogous manner to Example 1 (Steps 3 and 4) and Example 2, the title compound was prepared from (3-(indolin-1-yl)-2,3-dihydrobenzofuran-2-yl)methanol and methylamine. MS (ES) m/z 281.1.

### Example 11

### (+/−)-1-[3-(1H-indol-1-yl)-2,3-dihydro-1-benzofuran-2-yl]-N-methyl methanamine hydrochloride

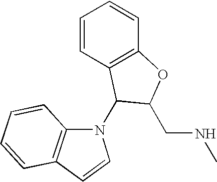

Step 1: A mixture of 3-(2-bromophenyl)-3-(2,3-dihydro-1H-indol-1-yl)propane-1,2-diol (1 g, 2.9 mmol, from Example 10, Step 2) and manganese dioxide (5 g, 57.4 mmol) in dichloromethane (20 mL) was stirred at room temperature for 12 hours. The reaction mixture was filtered through a plug of celite and the filtrate concentrated and purified on a silica gel column (ISCO, 0-100% ethyl acetate/hexane) to give 3-(2-bromophenyl)-3-(1H-indol-1-yl)propane-1,2-diol (0.87 g, 87%) as an off-white solid. MS (ES) m/z 345.8.

Step 2-4: In an analogous manner to Example 1 (Step 4) and Example 2, the title compound was prepared from (3-(1H-indol-1-yl)-2,3-dihydrobenzofuran-2-yl)methanol and methylamine. MS (ES) m/z 278.9; HRMS: calculated for C₁₈H₁₈N₂O+H+, 279.14919; found (ESI, [M+H]⁺), 279.1477.

### Example 12

### 7-fluoro-1-{(2S,3R)-7-fluoro-2-[(methylamino)methyl]-2,3-dihydro-1-benzofuran-3-yl}-3,3-dimethyl-1,3-dihydro-2H-indol-2-one hydrochloride

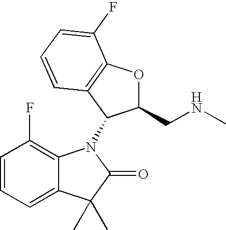

Step 1: A mixture of sodium perborate tetrahydrate (65 g, 422 mmol) in glacial acetic acid (250 mL) was stirred at 80° C. 2,6-Difluoroaniline (11.0 g, 85 mmol) in glacial acetic acid (50 mL) was added slowly to the mixture. The temperature was maintained between 80-90° C. for 1 hour. The cooled reaction mixture was poured into water and extracted twice with diethyl ether. The combined organic layers were washed with a dilute solution of sodium bicarbonate, dried over anhydrous magnesium sulfate and evaporated. The residue was purified via Biotage chromatography (FlasH90i, silica, 10% THF/hexane) and the product washed with hexane to afford 2,6-difluoronitrobenzene (7.0 g) (52%). MS (ESI) m/z 160 ([M+H]⁺).

Step 2: To a solution of 2,6-difluoronitrobenzene (5.0 g, 31.44 mmol) in dry N,N-dimethylformamide (50 mL) was added potassium carbonate (4.41 g, 32 mmol) and dimethylmalonate (3.6 mL, 31.44 mmol). The reaction mixture was heated to 65° C. and stirred for 24 hours. After cooling to room temperature, the mixture was neutralized with a dilute aqueous solution of hydrochloric acid and extracted with diethyl ether. The ethereal layer was dried over anhydrous magnesium sulfate, and concentrated in vacuo. Crystallization from 5% ethyl acetate/hexane gave 4.6 g (54%) 2-(6-fluoro-2-nitro-phenyl)-malonic acid dimethyl ester. MS (ESI) m/z 272 [M+H]⁺).

Step 3: 2-(6-Fluoro-2-nitro-phenyl)-malonic acid dimethyl ester (12 g, 44 mmol) in a 6N aqueous solution of hydrochloric acid (200 mL) was heated at reflux for 4 hours. The mixture was cooled, diluted with 250 mL of water and extracted with diethyl ether. The ethereal layer was dried over anhydrous magnesium sulfate, and concentrated in vacuo. Crystallization from 5% ethyl acetate/hexane gave 7.6 g of (6-fluoro-2-nitro-phenyl)-acetic acid (54%). MS (ESI) m/z 200 ([M+H]⁺).

Step 4: A mixture of (6-fluoro-2-nitro-phenyl)-acetic acid (9.6 g, 48 mmol) and 10% palladium on carbon (1.3 g) in acetic acid (100 ml) was hydrogenated at 50 psi for 24 hours. The catalyst was removed by filtration through Celite and the solvent was evaporated. The residue was then dissolved in ethanol (100 mL) and pyridinium para-toluenesulfonate (50 mg) was added and the mixture heated at reflux for 1 hour s. The mixture was cooled, poured into water, extracted with ethyl acetate and dried over anhydrous magnesium sulfate. The solvent was filtered and concentrated in vacuo. The solid was triturated with 5% ethyl acetate/hexane to give 6.0 g (83%) 7-fluoro-1,3-dihydro-indol-2-one. MS (ESI) m/z 152, [M+H]⁺).

Step 5: 7-Fluoro-1,3-dihydro-indol-2-one (7.3 g, 48 mmol) and lithium chloride (6.67 g, 158 mmol) were dissolved in tetrahydrofuran (200 mL). The solution was cooled to −78° C. and n-butyllithium (40 mL, 100 mmol) was added slowly over a 15 minute period. After 20 minutes at −78° C., methyl iodide (6 mL, 96 mmol) was added and the mixture allowed to warm to room temperature. After 24 hours, the mixture was poured into water and extracted with ethyl acetate. The organic layer was dried over anhydrous magnesium sulfate, and concentrated in vacuo. The crude product was purified via Biotage chromatography (Flash40i, silica, 10% then 20% ethyl acetate/hexane) gave 4.1 g (48%) 7-fluoro-3,3-dimethyl-1,3-dihydro-2H-indol-2-one. MS (ESI) m/z 180, ([M+H]⁺).

Step 6: In an analogous manner to patent application example 1, step 2, (2E)-3-(2,3-difluorophenyl)prop-2-en-1-ol was prepared from (2E)-3-(2,3-difluorophenyl)acrylic acid. MS (ES) m/z 152.8 [M−H₂O]⁺).

A mixture of (2E)-3-(2,3-difluorophenyl)prop-2-en-1-ol (1.75 g, 10.3 mmol), m-chloroperbenzoic acid (3.5 mL, 15.4 mmol), and saturated aqueous sodium carbonate (2 mL) in methylene chloride (35 mL) was stirred at room temperature for 16 hours. The mixture was treated with a saturated sodium bicarbonate solution (50 mL) and extracted with ethyl acetate (50 mL). The organic layer was dried over anhydrous sodium sulfate and evaporated onto silica gel. The crude product was purified via Isco (0-100% ethyl acetate/hexane) to give [3-(2,3-difluorophenyl)oxiran-2-yl]methanol as a white solid (1.44 g, 76%).

Step 7: To a solution of 7-fluoro-3,3-dimethyl-1,3-dihydro-2H-indol-2-one (0.71 g, 4 mmol) in dimethylformamide (1 mL) was added 1M lithium hexamethyldisilazide in tetrahydrofuran (10 mL, 10 mmol), while maintaining the reaction temperature below 20° C. Upon complete addition, the reaction was stirred for 20 minutes. In a separate flask, [3-(2,3-difluorophenyl)oxiran-2-yl]methanol (0.78 g, 4.2 mmol) and titanium isopropoxide (1.2 mL, 4.2 mmol) in tetrahydrofuran (10 mL) were prepared and this solution added to the oxindole solution. After 3 hours, the reaction mixture was partitioned between saturated aqueous ammonium chloride (100 mL) and ethyl acetate (100 mL). 2N hydrochloric acid (20 mL) was added in order to break up the emulsion. The organic layers were dried over anhydrous sodium sulfate and concentrated on silica gel. The crude product was purified via Isco (20-100% ethyl acetate/hexane) to give 1-[1-(2,3-difluorophenyl)-2,3-dihydroxypropyl]-7-fluoro-3,3-dimethyl-1,3-dihydro-2H-indol-2-one as a yellow amorphous solid (1.3 g, 89%). MS (ES) m/z 366.1 [M+H]⁺.

Step 8: To a solution of 1-[1-(2,3-difluorophenyl)-2,3-dihydroxypropyl]-7-fluoro-3,3-dimethyl-1,3-dihydro-2H-indol-2-one (0.66 g, 1.8 mmol) in tetrahydrofuran (60 mL) was added potassium tert-butoxide (0.43 g, 3.6 mmol) and the reaction heated to 70° C. for 2 hours. The reaction was partitioned between saturated aqueous ammonium chloride (50 mL) and ethyl acetate (50 mL). The organic layers were dried over anhydrous sodium sulfate and concentrated on silica gel. The crude product was purified via Isco (silica gel, 0-100% ethyl acetate/hexane) to give 7-fluoro-1-[7-fluoro-2-(hydroxymethyl)-2,3-dihydro-1-benzofuran-3-yl]-3,3-dimethyl-1,3-dihydro-2H-indol-2-one as a yellow solid (0.25 g, 40%). MS (ES) m/z 346.2 ([M+H]+).

Step 9: To 7-fluoro-1-[7-fluoro-2-(hydroxymethyl)-2,3-dihydro-1-benzofuran-3-yl]-3,3-dimethyl-1,3-dihydro-2H-indol-2-one (0.14 g, 0.41 mmol) and pyridine (3 mL) was added tosyl chloride (0.085 g, 0.45 mmol) and the reaction stirred overnight. The reaction was poured into 2N hydrochloric acid (25 mL) and extracted with ethyl acetate (25 mL). The organics were washed with saturated aqueous sodium bicarbonate (25 mL) followed by saturated ammonium chloride (25 mL). The organic layers were dried over anhydrous sodium sulfate and used in the next step without further purification. The tosylate (0.52 g, 1 mmol) was treated with and excess of 33% methylamine in methanol in a sealed tube and heated to 80° C. for 1 hour. The reaction was poured into saturated aqueous sodium bicarbonate (25 mL) and extracted with ethyl acetate (25 mL). The organic layers were dried over anhydrous sodium sulfate and concentrated on silica gel. The crude product was purified via Isco (silica gel, 80% ethyl acetate/hexane) to give racemic 7-fluoro-1-{7-fluoro-2-[(methylamino)methyl]-2,3-dihydro-1-benzofuran-3-yl}-3,3-dimethyl-1,3-dihydro-2H-indol-2-one. The racemate was separated via preparatory SFC using a Chiralpak AD-H column (10% methanol with 0.2% DMEA). Treatment of pure with 1N hydrochloric acid in ether gave the title compound, 7-fluoro-1-{(2S,3R)-7-fluoro-2-[(methylamino)methyl]-2,3-dihydro-1-benzofuran-3-yl}-3,3-dimethyl-1,3-dihydro-2H-indol-2-one as a white salt (0.013 g, 8%); MS (ES) m/z 359.2 [M+H]⁺.

### Example 13

### 1-[(2R,3S)-5-fluoro-3-(7-fluoro-3,3-dimethyl-2,3-dihydro-1H-indol-1-yl)-2,3-dihydro-1-benzofuran-2-yl]-N-methylmethanamine hydrochloride

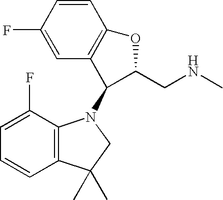

Steps 1: In an analogous manner to Example 1, step 2, [(2R,3R)-3-(2,5-difluorophenyl)oxiran-2-yl]methanol was prepared from (E)-3-(2,5-difluorophenyl)acrylic acid as a white solid. MS (ES) m/z 186.8.

Step 2: In an analogous manner to Example 1, step 3, (2S,3S)-3-(2,5-difluorophenyl)-3-(7-fluoro-3,3-dimethyl-2,3-dihydro-1H-indol-1-yl)propane-1,2-diol was prepared from 7-fluoro-3,3-dimethylindoline and [(2R,3R)-3-(2,5-difluorophenyl)oxiran-2-yl]methanol as a white solid. MS (ES) m/z 351.8.

Step 3: In an analogous manner to Example 12, step 8, [(2S,3S)-5-fluoro-3-(7-fluoro-3,3-dimethyl-2,3-dihydro-1H-indol-1-yl)-2,3-dihydro-1-benzofuran-2-yl]methanol was prepared from (2S,3S)-3-(2,5-difluorophenyl)-3-(7-fluoro-3,3-dimethyl-2,3-dihydro-1H-indol-1-yl)propane-1,2-diol as a clear oil. MS (ESI) m/z 332.1.

Step 4: To a solution of [(2S,3S)-5-fluoro-3-(7-fluoro-3,3-dimethyl-2,3-dihydro-1H-indol-1-yl)-2,3-dihydro-1-benzofuran-2-yl]methanol (0.4 g, 1.21 mmol), N-methyl-2-nitrobenzenesulfonamide (0.33 g, 1.45 mmol), triphenylphosphine (0.41 g, 1.45 mmol) in THF (10 mL) was added diisopropyl azodicarboxylate (0.3 mL, 1.45 mmol) at room temperature under nitrogen. The reaction mixture was stirred at room temperature for 18 hours concentrated in vacuo. The residue was purified on a silica gel column (hexane:acetate=3:1) to afford N-{[(2R,3S)-5-fluoro-3-(7-fluoro-3,3-dimethyl-2,3-dihydro-1H-indol-1-yl)-2,3-dihydro-1-benzofuran-2-yl]methyl}-N-methyl-2-nitrobenzenesulfonamide as a yellowish gum (0.5 g, 79%). MS (ES) m/z 529.7.

Step 5: To a solution of N-[(2R,3S)-5-fluoro-3-(7-fluoro-3,3-dimethyl-2,3-dihydro-1H-indol-1-yl)-2,3-dihydro-1-benzofuran-2-yl]methyl}-N-methyl-2-nitrobenzenesulfonamide (0.5 g, 0.9 mmol) in DMF (5 mL) was added at room temperature under nitrogen potassium carbonate (0.37 g, 2.7 mmol) and thiophenol (0.1 mL, 1.4 mmol). After stirred for 18 hours, the reaction mixture was poured into a solution of saturated ammonium chloride (50 mL) and extracted with ethyl acetate (2×50 mL). The organic layers were combined, dried over sodium sulfate, concentrated, and purified on silica gel column (ISCO 0-30% methanol/methylene chloride) to give the title compound, 1-[(2R,3S)-5-fluoro-3-(7-fluoro-3,3-dimethyl-2,3-dihydro-1H-indol-1-yl)-2,3-dihydro-1-benzofuran-2-yl]-N-methylmethanamine which was converted to its HCl salt as an off-white solid (0.26 g, 76%). [α]_{D}²⁵=+130.4° (c=1%, MeOH); MS (ES) m/z 344.9 [M+H]⁺; HRMS: calcd for C₂₀H₂₂F₂N₂O+H⁺, 345.17729; found (ESI, [M+H]⁺ Obs'd), 345.1775.

### Example 14

### 1-{6-fluoro-2-[(methylamino)methyl]-2,3-dihydro-1-benzofuran-3-yl}-3,3-dimethyl-1,3-dihydro-2H-indol-2-one hydrochloride

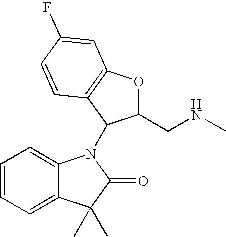

Step 1: In an analogous manner to example 1, steps 4 and 5, 2-nitrophenylacetic acid was converted to 3,3-dimethyl-1,3-dihydro-2H-indol-2-one. MS (ES) m/z 162.1.

Step 2: In an analogous manner to example 12, step 6, [3-(2,4-difluorophenyl)oxiran-2-yl]methanol was prepared from (2E)-3-(2,4-difluorophenyl)acrylic acid. MS (ES) m/z 169.1.

Step 3: In an analogous manner to example 12, step 7, 3,3-dimethyl-1,3-dihydro-2H-indol-2-one (0.55 g, 3.4 mmol) was treated with [3-(2,4-difluorophenyl)oxiran-2-yl]methanol (0.82 g, 3.6 mmol) to give 1-[1-(2,4-difluorophenyl)-2,3-dihydroxypropyl]-3,3-dimethyl-1,3-dihydro-2H-indol-2-one as an amorphous solid (0.91 g, 76%). MS (ES) m/z 347.7.

Step 4: In an analogous manner to example 12, step 8, 1-[1-(2,4-difluorophenyl)-2,3-dihydroxypropyl]-3,3-dimethyl-1,3-dihydro-2H-indol-2-one (0.51 g, 1.5 mmol) was treated with potassium tert butoxide (0.35 g, 3 mmol) to give [6-fluoro-2-(hydroxymethyl)-2,3-dihydro-1-benzofuran-3-yl]-3,3-dimethyl-1,3-dihydro-2H-indol-2-one (0.25 g, 52%). MS (ES) m/z 327.2.

Step 5. In an analogous manner to example 12, step 9, [6-fluoro-2-(hydroxymethyl)-2,3-dihydro-1-benzofuran-3-yl]-3,3-dimethyl-1,3-dihydro-2H-indol-2-one (0.19 g, 0.58 mmol) was converted to a tosylate and treated with an excess of 33% methylamine in ethanol, followed by treatment with 1N hydrochloric acid in ether to give 1-{6-fluoro-2-[(methylamino)methyl]-2,3-dihydro-1-benzofuran-3-yl}-3,3-dimethyl-1,3-dihydro-2H-indol-2-one HCl salt as an off-white salt (0.087 g, 40%). MS (ES) m/z 341.2 [M+H]⁺.

### Example 15

### 3,3-dimethyl-1-{2-[(methylamino)methyl]-2,3-dihydro-1-benzofuran-3-yl}-1,3-dihydro-2H-indol-2-one hydrochloride

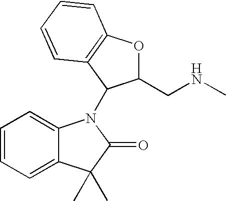

Step 1: In an analogous manner to patent example 12, step 6, [3-(2-fluorophenyl)oxiran-2-yl]methanol was prepared from (2E)-3-(2-fluorophenyl)acrylic acid. MS (ES) m/z 168.0.

Step 2. In an analogous manner to example 12, step 7, 3,3-dimethyl-1,3-dihydro-2H-indol-2-one (1.62 g, 10 mmol) was treated with [3-(2-fluorophenyl)oxiran-2-yl]methanol (1.8 g, 11 mmol) to give 1-[1-(2-fluorophenyl)-2,3-dihydroxypropyl]-3,3-dimethyl-1,3-dihydro-2H-indol-2-one as a yellow oil (2.7 g, 82%). MS (ES) m/z 330.0.

Step 3. In an analogous manner to example 12, step 8, 1-[1-(2-fluorophenyl)-2,3-dihydroxypropyl]-3,3-dimethyl-1,3-dihydro-2H-indol-2-one (2.6 g, 7.9 mmol) was treated with potassium tert-butoxide (1.9 g, 15.8 mmol) to give 1-(2-(hydroxymethyl)-2,3-dihydro-1-benzofuran-3-yl)-3,3-dimethyl-1,3-dihydro-2H-indol-2-one. MS (ES) m/z 309.1.

Step 4. In an analogous manner to example 12, step 9, 1-(2-(hydroxymethyl)-2,3-dihydro-1-benzofuran-3-yl)-3,3-dimethyl-1,3-dihydro-2H-indol-2-one (0.6 g, 1.9 mmol) was converted to a tosylate and treated with an excess of 33% methylamine in ethanol, followed by treatment with 1N hydrochloric acid in ether to give 3,3-dimethyl-1-{2-[(methylamino)methyl]-2,3-dihydro-1-benzofuran-3-yl}-1,3-dihydro-2H-indol-2-one HCl salt as an off-white salt (0.086 g, 13%). MS (ES) m/z 322.6; HRMS: calcd for C₂₀H₂₂N₂O₂+H⁺, 323.17540; found (ESI, [M+H]⁺ Obs'd), 323.1759.

### Example 16

### 1-[3-(3,4-dihydroquinolin-1(2H)-yl)-2,3-dihydro-1-benzofuran-2-yl]-N-methylmethanamine hydrochloride

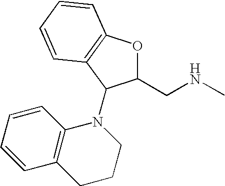

Step 1: To a solution of [3-(2-fluorophenyl)oxiran-2-yl]methanol (Example 15, step 1, 2.71 g, 16.1 mmol) and titanium isopropoxide (5.8 mL, 19.3 mmol) in dichloromethane (50 mL) was added commercially available 1,2,3,4-tetrahydroquinoline (2.1 g, 16.1 mmol) dissolved in dichloromethane (5 mL). After stirred for 16 hours, the reaction mixture was poured into 1N hydrochloric acid (25 mL) and extracted with ethyl acetate (25 mL). The organic layer was washed with sodium bicarbonate (25 mL), dried using anhydrous sodium sulfate, and concentrated to give 3-(3,4-dihydroquinolin-1(2H)-yl)-3-(2-fluorophenyl)propane-1,2-diol (2.73 g, 56%). MS (ES) m/z 2301.1.

Step 2. In an analogous manner to example 12, step 8, 3-(3,4-dihydroquinolin-1(2H)-yl)-3-(2-fluorophenyl)propane-1,2-diol (2.73 g, 9.1 mmol) was treated with potassium tert butoxide (2.7 g, 22.6 mmol) to give [3-(3,4-dihydroquinolin-1(2H)-yl)-2,3-dihydro-1-benzofuran-2-yl]methanol (1.73 g, 75%). MS (ES) m/z 282.1;

Step 3: To a solution of [3-(3,4-dihydroquinolin-1(2H)-yl)-2,3-dihydro-1-benzofuran-2-yl]methanol (0.17 g, 0.6 mmol), N-methyl-1-(2-nitrophenylsulfonyl)methanamine (0.14 g, 0.66 mmol), and triphenylphosphine (0.34 g, 1.3 mmol) in tetrahydrofuran (15 mL) was added at room temperature diisopropylazodicarboxylate (0.26 mL, 1.3 mmol) dropwise. After stirred for 4 hours under nitrogen, the reaction mixture was poured into saturated aqueous ammonium chloride (25 mL) and extracted with ethyl acetate (2×25 mL). The organic layers were dried over sodium sulfate and concentrated onto silica gel. The crude product was purified via Isco (silica gel, ethyl acetate/hexane 0-100%) to give N-((3-(3,4-dihydroquinolin-1(2H)-yl)-2,3-dihydrobenzofuran-2-yl)methyl)-N-methyl-2-nitrobenzenesulfonamide. MS (ES) m/z 479.1.

To N-((3-(3,4-dihydroquinolin-1(2H)-yl)-2,3-dihydrobenzofuran-2-yl)methyl)-N-methyl-2-nitrobenzenesulfonamide (0.29 g, 0.6 mmol) in dimethylformamide (4 mL) was added potassium carbonate (0.25 g, 1.8 mmol) and thiophenol (0.065 mL, 0.63 mmol). After stirred for 1 hour, the reaction mixture was poured into saturated ammonium chloride (25 mL) and extracted with using ethyl acetate (2×25 mL). The organic layers were dried over sodium sulfate, concentrated onto silica gel, and purified via Isco (silica gel, ethyl acetate/hexane, 0-100%; methanol/dichloromethane 0-20%) to afford the title compound as a free base. Treatment of free base with 1N hydrochloric acid in ether gave 1-[3-(3,4-dihydroquinolin-1(2H)-yl)-2,3-dihydro-1-benzofuran-2-yl]-N-methylmethanamine HCl salt as an off-white solid (0.023 g, 3%). MS (ES) m/z 295.1 [M=H]⁺.

### Example 17

### N-methyl-2-[(methylamino)methyl]-N-phenyl-2,3-dihydro-1-benzofuran-3-amine hydrochloride

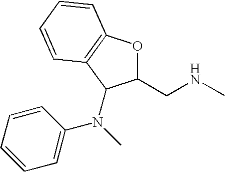

Step 1: In an analogous manner to example 16, step 1, N-methylaniline (1.8 g, 16.7 mmol) was treated with [3-(2-fluorophenyl)oxiran-2-yl]methanol (2.8 g, 16.7 mmol) to give 3-(2-fluorophenyl)-3-(methyl(phenyl)amino)propane-1,2-diol (1.27 g, 28%). MS (ES) m/z 275.1.

Step 2. In an analogous manner to example 12, step 8, 3-(2-fluorophenyl)-3-(methyl(phenyl)amino)propane-1,2-diol (1.27 g, 4.6 mmol) was treated with potassium tert butoxide (1.1 g, 9.2 mmol) to give 3-[methyl(phenyl)amino]-2,3-dihydro-1-benzofuran-2-yl}methanol as a clear oil (0.53 g, 44%). MS (ESI) m/z 256.

Step 3: In an analogous manner to example 16, step 3, 3-[methyl(phenyl)amino]-2,3-dihydro-1-benzofuran-2-yl}methanol (0.1 g, 0.39 mmol) was converted to N-methyl-2-[(methylamino)methyl]-N-phenyl-2,3-dihydro-1-benzofuran-3-amine HCl salt as an off-white solid (0.015 g, 13%). MS (ES) m/z 269.1 [M=H]⁺.

### Example 18

### 4-Fluoro-3-{(2SR,3RS)-6-fluoro-2-[(methylamino)methyl]-2,3-dihydro-1-benzofuran-3-yl}-1-isopropyl-1,3-dihydro-2H-benzimidazol-2-one hydrochloride

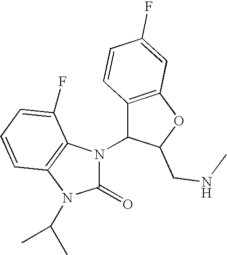

Step 1: To a solution of 2,6-difluoronitrobenzene (6.37 g, 40.0 mmol) in triethylamine (70 mL) under nitrogen was added dropwise isopropylamine (4.3 mL, 50 mmol, 12.5 equiv.) via an addition funnel. The reaction mixture was stirred at room temperature overnight. All volatiles were removed under reduced pressure, and the residue was dissolved in ethanol (50 mL). Palladium on carbon powder (10 wt %, ˜0.4 g) was added, and the mixture was stirred under hydrogen atmosphere (20 psi) for 30 min. The reaction mixture was filtered through Celite and concentrated to dryness. The crude black liquid residue was purified by Isco CombiFlash Companion column chromatography (silica gel, 120-g column, 0-15% ethyl acetate/hexane) and the resulting purple solid was recrystallized (warm hexane/−25° C.) to give pure 3-fluoro-N-isopropylbenzene-1,2-diamine as light purple crystals. Yield: 4.21 g (63%). MS (ES) m/z 168.7 ([M+H]⁺); HRMS: calcd for C₉H₁₃FN₂+H⁺, 169.1136; found (ESI, [M+H]⁺), 169.1139.

Step 2: To a solution of 3-fluoro-N-isopropylbenzene-1,2-diamine (2.02 g, 12.0 mmol) in dichloromethane (50 mL) was added slowly a solution of triphosgene (1.19 g, 4.0 mmol, ⅓ equiv.) in dichloromethane (20 mL). The reaction mixture was stirred overnight, then poured into water (100 mL) and extracted. The organic layer was washed with water, brine, dried (anhydrous Na₂SO₄), filtered, and concentrated to give a white solid, which was recrystallized (warm chloroform/hexane/−25° C.) to give pure 4-fluoro-1-isopropyl-1,3-dihydro-2H-benzimidazol-2-one as white crystals. Yield: 1.56 g (67%). MS (ES) m/z 195.2 ([M+H]⁺).

Step 3: In an analogous manner to Example 12, Step 7, 3-(1RS,2RS)[1-(2,4-difluorophenyl)-2,3-dihydroxypropyl]-4-fluoro-1-isopropyl-1,3-dihydro-2H-benzimidazol-2-one was prepared from 4-fluoro-1-isopropyl-1,3-dihydro-2H-benzimidazol-2-one and (2RS,3RS)-[3-(2,4-difluorophenyl)oxiran-2-yl]methanol (Example 1, Step 2) as white needles. MS (ES) m/z 381.0 ([M+H]⁺); HRMS: calcd for C₁₉H₁₉F₃N₂O₃+H⁺, 381.1421; found (ESI, [M+H]⁺), 381.1420.

Step 4: In an analogous manner to Example 12, Step 8, 4-fluoro-3-[(2RS 3RS)-6-fluoro-2-(hydroxymethyl)-2,3-dihydro-1-benzofuran-3-yl]-1-isopropyl-1,3-dihydro-2H-benzimidazol-2-one was prepared from 3-(1RS,2RS)[1-(2,4-difluorophenyl)-2,3-dihydroxypropyl]-4-fluoro-1-isopropyl-1,3-dihydro-2H-benzimidazol-2-one as a white foam. MS (ES) m/z 360.9 ([M+H]⁺); HRMS: calcd for C₁₉H₁₈F₂N₂O₃+H⁺, 361.1358; found (ESI, [M+H]⁺), 361.1363.

Step 5: In an analogous manner to Example 12, Step 9, 4-fluoro-3-{(2SR,3RS)-6-fluoro-2-[(methylamino)methyl]-2,3-dihydro-1-benzofuran-3-yl}-1-isopropyl-1,3-dihydro-2H-benzimidazol-2-one hydrochloride was prepared from 4-fluoro-3-[(2RS,3RS)-6-fluoro-2-(hydroxymethyl)-2,3-dihydro-1-benzofuran-3-yl]-1-isopropyl-1,3-dihydro-2H-benzimidazol-2-one as a white powder. MS (ES) m/z 374.0 ([M+H]⁺); HRMS: calcd for C₂₀H₂₁F₂N₃O₂+H⁺, 374.1675; found (ESI, [M+H]⁺), 374.1682.

### Example 19

### 4-Fluoro-3-{(2S,3R)-6-fluoro-2-[(methylamino)methyl]-2,3-dihydro-1-benzofuran-3-yl}-1-isopropyl-1,3-dihydro-2H-benzimidazol-2-one hydrochloride

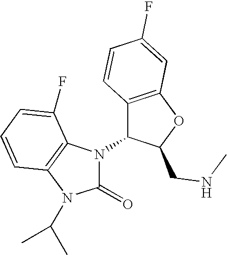

Step 1: Racemic 4-fluoro-3-{(2SR,3RS)-6-fluoro-2-[(methylamino)methyl]-2,3-dihydro-1-benzofuran-3-yl}-1-isopropyl-1,3-dihydro-2H-benzimidazol-2-one hydrochloride (Example 18) was dissolved in methanol (40 mg/5 mL). The resulting solution was stack injected onto the Supercritical Fluid Chromatography instrument at 0.4 mL increments. The baseline resolved enantiomers, using the conditions described below, were collected. The enantiomeric purity of each enantiomer was determined under similar Supercritical Fluid Chromatography conditions using a Chiralcel OJ-H 5u, 250 mm L×4.6 mm ID column at 2 mL/min flow rate using Analytical Supercritical Fluid Chromatography (Berger Instruments, Inc. Newark, Del. USA). Each enantiomer displayed >99.9% chiral purity.

SFC Instrument: Berger MultiGram Prep SFC (Berger Instruments, Inc. Newark, Del. 19702.

Column: Chiralcel AD-H; 5p; 250 mm L×20 mm ID (Chiral Technologies, Inc., Exton, Pa., USA)

Column temperature: 35° C.

SFC Modifier: 20% MeOH with 0.2% DMEA/80% CO₂

Flow rate: 50 mL/min

Outlet Pressure: 100 bar

Detector: UV at 220 nm

Step 2: The free base 4-fluoro-3-{(2S,3R)-6-fluoro-2-[(methylamino)methyl]-2,3-dihydro-1-benzofuran-3-yl}-1-isopropyl-1,3-dihydro-2H-benzimidazol-2-one, which was isolated as Peak 1 of the chiral separation (step 1), was dissolved in a minimal amount of methanol. Hydrogen chloride (1.0 M in ethyl ether, ˜2 equiv.) was added with swirling. All volatiles were then removed under reduced pressure, and the resulting white solid was re-dissolved in a minimal amount of warm methanol. Ethyl ether was added until the solution became slightly cloudy. The mixture was cooled to −25° C. for several hours. White solid formed was collected by decantation, washed with ethyl ether and dried in vacuo to give pure 4-fluoro-3-{(2S,3R)-6-fluoro-2-[(methylamino)methyl]-2,3-dihydro-1-benzofuran-3-yl}-1-isopropyl-1,3-dihydro-2H-benzimidazol-2-one hydrochloride. Absolute stereochemistry was arbitrarily assigned. Chiral purity: 99.9%. MS (ES) m/z 374.0 ([M+H]⁺); HRMS: calcd for C₂₀H₂₁F₂N₃O₂+H⁺, 374.1675; found (ESI, [M+H]⁺), 374.1681.

### Example 20

### 4-Fluoro-3-{(2R,3S)-6-fluoro-2-[(methylamino)methyl]-2,3-dihydro-1-benzofuran-3-yl}-1-isopropyl-1,3-dihydro-2H-benzimidazol-2-one hydrochloride

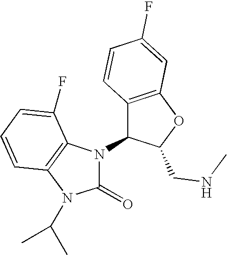

In an analogous manner to Example 20, step 2, 4-fluoro-3-{(2R,3S)-6-fluoro-2-[(methylamino)methyl]-2,3-dihydro-1-benzofuran-3-yl}-1-isopropyl-1,3-dihydro-2H-benzimidazol-2-one hydrochloride was prepared from 4-fluoro-3-{(2R,3S)-6-fluoro-2-[(methylamino)methyl]-2,3-dihydro-1-benzofuran-3-yl}-1-isopropyl-1,3-dihydro-2H-benzimidazol-2-one, which was isolated as Peak 2 of the chiral separation (Example 19, step 1). Absolute stereochemistry was arbitrarily assigned. Chiral purity: 99.2%. MS (ES) m/z 374.0 ([M+H]⁺); HRMS: calcd for C₂₀H₂₁F₂N₃O₂+H⁺, 374.1675; found (ESI, [M+H]⁺), 374.1683.

### Cell Lines, Culture Reagents, and Assays

MDCK-Net6 cells, stably transfected with human hNET (Pacholczyk, T., R. D. Blakely, and S. G. Amara, Nature, 1991, 350(6316): p. 350-4) were cultured in growth medium containing high glucose DMEM (Gibco, Cat. No. 11995), 10% FBS (dialyzed, heat-inactivated, US Bio-Technologies, Lot FBD1129HI) and 500 μg/ml G418 (Gibco, Cat. No. 10131). Cells were plated at 300,000 T75 flask and cells were split twice weekly.

### Functional Norepinephrine (NE) Uptake Assay

On day 1, cells were plated at 3,000 cells/well in growth medium and maintained in a cell incubator (37° C., 5% CO₂). On day 2, growth medium was replaced with 200 μl of assay buffer (25 mM HEPES; 120 mM NaCl; 5 mM KCl; 2.5 mM CaCl₂; 1.2 mM MgSO₄; 2 mg/ml glucose (pH 7.4, 37° C.)) containing 0.2 mg/ml ascorbic acid and 1 μM pargyline. For screening, 25 μl of compound in 4% DMSO was added directly to each well and the plate is incubated for 5 minutes at 37° C.

To initiate the norepinephrine reuptake, 16 nM (final concentration) of ³H norepinephrine (specific activity; 40-80 Ci/mmol) in assay buffer was delivered in 25 μl aliquots to each well, and the plates were incubated for 5 minutes at 37° C. The reaction was aspirated from the plate and the cells washed with 250 μl of 50 mM Tris Buffer (4° C.). The plates were left to dry for 1 hour. The cells were lysed using 0.25 M NaOH solution then placed on a shake table and vigorously shaken for 10 minutes. After cell lysis, 100 μl of Microscint 20 (PerkinElmer; #87-051101) were added to the plates and the plates were sealed with film tape and replaced on the shake table for a minimum of 10 minutes. The plates were counted in a TopCount counter (PerkinElmer).

### Evaluation of Results

For screening single point determinations, each compound plate contains at least 3 control wells (maximum NE reuptake determinant) and 3 non-specific wells determined by adding 20 μM of desipramine (minimum NE reuptake determinant). Determination of active compounds are calculated using a Microsoft Excel spread sheet applying the following formula:

% inhibition=[1−((mean cpm test compound wells−mean cpm non-specific wells)/(mean cpm control wells−mean cpm non-specific wells))]×100

For IC₅₀ determination, raw cpm values were generated in a data file from the TopCount counter. The data was organized using Microsoft Excel and transferred into PRIZM graphing and statistical program, which calculates the estimated IC₅₀ value. Calculation of IC₅₀ values were made using non-linear regression analysis with a sigmoidal dose response with variable slope. The statistical program used wells containing ³H norepinephrine only as the maximal NE reuptake determinant and wells containing ³H norepinephrine plus 20 μM desipramine as the minimal NE reuptake determinant (non-specific determinant). Estimation of the IC₅₀ value was completed on a log scale and the line was fit between the maximal and minimal NE reuptake values. In the event that the highest test concentration does not exceed 50% reuptake inhibition, data will be reported as percent maximal NE reuptake at the highest concentration tested. The results are reported in Table 1.

TABLE 1

| | | hNET IC₅₀ (nM) |
|---|---|---|
| Example | hNET IC₅₀ (nM) | (% Inhibition) |
| 1 | 27.2 | — |
| 2 | 343 | — |
| 3 | 319 | — |
| 4 | 95 | — |
| 5 | — | 63% @ 6 μM |
| 6 | — | 30% @ 6 μM |
| 7 | — | 37% @ 6 μM |
| 8 | — | 42% @ 6 μM |
| 9 | 846 | — |
| 10 | 244 | — |
| 11 | 301 | 42% @ 6 μM |
| 12 | 1028 | |
| 13 | 88.5 | |
| 14 | | 54% @ 6 μM |
| 15 | 1135 | |
| 16 | 811 | |
| 17 | 3551 | |
| 18 | 3797 | |
| 19 | 12400 | |
| 20 | 1750 | |

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A compound of formula I: 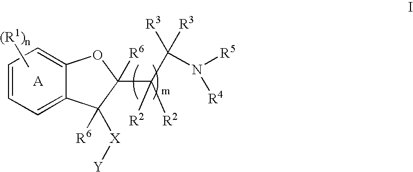
or a pharmaceutically acceptable salt thereof;
wherein:
m is an integer from 0 to 3;
n is an integer from 0 to 4;
X is O, S, SO₂, or NR⁷;
Y is aryl substituted with 0-3 R¹ or heteroaryl substituted with 0-3 R¹;
R¹ is, independently at each occurrence, alkyl, alkoxy, halo, CF₃, OCF₃, arylalkoxy substituted with 0-3 R⁸, hydroxy, alkanoyloxy, nitro, nitrile, alkenyl, alkynyl, alkylsulfoxide, arylsulfoxide substituted with 0-3 R⁸, alkylsulfone, arylsulfone substituted with 0-3 R⁸, alkylsulfonamide, arylsulfonamide substituted with 0-3
R⁸, heteroarylsulfonamide substituted with 0-3 R⁸, heteroarylmethoxy substituted with 0-3 R⁸, alkylamido, or arylamido substituted with 0-3 R⁸; or
two adjacent R¹ also represent methylenedioxy;
R² is H, F, C₁-C₄ alkyl, or OR⁹;
R³ is H, F, C₁-C₄ alkyl, or OR¹⁰;
R⁴ is H, C₁-C₄ alkyl, arylalkyl substituted with 0-3 R¹¹, heteroarylalkyl substituted with 0-3 R¹¹, cycloheptylmethyl, cyclohexylmethyl, cyclopentylmethyl, or cyclobutylmethyl; or
R² and R⁴, together with the nitrogen and carbon through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
R³ and R⁴, together with the nitrogen and carbon through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
R⁵ is H, C₁-C₄ alkyl, arylalkyl substituted with 0-3 R¹², heteroarylalkyl substituted with 0-3 R¹², cycloheptylmethyl, cyclohexylmethyl, cyclopentylmethyl, or cyclobutylmethyl; or
R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃;
R⁶ is H, alkyl, or perfluoroalkyl;
R⁷ is H, alkyl, or aryl substituted with 0-3 R¹³; or
R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃;
R⁸, R¹¹, R¹², and R¹³ are, independently at each occurrence, alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or
two adjacent R⁸, or two adjacent R¹¹, or two adjacent R¹², or two adjacent R¹³ also represent methylenedioxy;
R⁹ and R¹⁰ are, independently at each occurrence H or C₁-C₄ alkyl; and
wherein
1-3 carbon atoms in ring A may optionally be replaced with N.

2. A compound according to claim 1,
wherein:
X is O.

3. A compound according to claim 1,
wherein:
X is S or SO₂.

4. A compound according to claim 1,
wherein:
n is an integer from 0 to 2.

5. A compound according to claim 1,
wherein:
m is an integer from 1 to 2.

6. A compound according to claim 1,
wherein:
R¹ is, independently at each occurrence, C₁-C₆ alkyl, alkoxy, halo, CF₃, OCF₃, nitrile, or aryl substituted with 0-3 R⁸.

7. A compound according to claim 1,
wherein:
R¹ is, independently at each occurrence, methyl, methoxy, fluoro, chloro, bromo, CF₃, OCF₃, nitrile, or phenyl.

8. A compound according to claim 1,
wherein:
R² is, independently at each occurrence, H, fluoro, methyl, ethyl, hydroxy, or methoxy.

9. A compound according to claim 1,
wherein:
R² is, independently at each occurrence, hydroxy.

10. A compound according to claim 1,
wherein:
R³ is, independently at each occurrence, H, fluoro, methyl, ethyl, or methoxy.

11. A compound according to claim 1,
wherein:
R³ is, independently at each occurrence, H.

12. A compound according to claim 1,
wherein:
R⁴ is H or C₁-C₄ alkyl.

13. A compound according to claim 1,
wherein:
R⁴ is H or methyl.

14. A compound according to claim 1,
wherein:
R⁵ is H or C₁-C₄ alkyl.

15. A compound according to claim 1,
wherein:
R⁵ is H or methyl.

16. A compound according to claim 1,
wherein:
R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃.

17. A compound according to claim 1,
wherein:
R² and R⁴, together with the nitrogen and the carbon through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃.

18. A compound according to claim 1,
wherein:
R³ and R⁴, together with the nitrogen and the carbon through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃.

19. A compound according to claim 1,
wherein:
R⁶ is, independently at each occurrence, H, methyl, ethyl, or perfluoromethyl.

20. A compound according to claim 1,
wherein:
R⁶ is, independently at each occurrence, H.

21. A compound according to claim 1,
wherein:
X is NR₇.

22. A compound according to claim 1,
wherein:
R⁷ is H, methyl, ethyl or phenyl.

23. A compound according to claim 1,
wherein:
R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃.

24. A compound according to claim 1,
wherein:
R⁷ and Y, together with the nitrogen through which they are attached, form oxoindolyl, benzimidazolonyl, indolinyl, or indolyl optionally substituted with C₁-C₄ alkyl, F, or CF₃.

25. A compound according to claim 1, selected from the group consisting of:
1-[3-(7-fluoro-3,3-dimethyl-2,3-dihydro-1H-indol-1-yl)-2,3-dihydro-1-benzofuran-2-yl]-N-methylmethanamine;
1-[3-(5-fluoro-3,3-dimethyl-2,3-dihydro-1H-indol-1-yl)-2,3-dihydro-1-benzofuran-2-yl]-N-methylmethanamine;
1-[3-(7-fluoro-3,3-dimethyl-2,3-dihydro-1H-indol-1-yl)-2,3-dihydro-1-benzofuran-2-yl]-N,N-dimethylmethanamine;
1-[3-(7-fluoro-3,3-dimethyl-2,3-dihydro-1H-indol-1-yl)-2,3-dihydro-1-benzofuran-2-yl]methanamine;
1-[3-(7-fluoro-3,3-dimethyl-2,3-dihydro-1H-indol-1-yl)-2,3-dihydro-1-benzofuran-2-yl]-N-methylmethanamine;
N-{[3-(7-fluoro-3,3-dimethyl-2,3-dihydro-1H-indol-1-yl)-2,3-dihydro-1-benzofuran-2-yl]methyl}propan-2-amine;
1-[3-(7-fluoro-3,3-dimethyl-2,3-dihydro-1H-indol-1-yl)-2,3-dihydro-1-benzofuran-2-yl]-N,N-dimethylmethanamine;
N-{[3-(7-fluoro-3,3-dimethyl-2,3-dihydro-1H-indol-1-yl)-2,3-dihydro-1-benzofuran-2-yl]methyl}ethanamine;
1-[3-(2-ethoxyphenoxy)-2,3-dihydro-1-benzofuran-2-yl]-N-methylmethanamine;
1-[3-(2,3-dihydro-1H-indol-1-yl)-2,3-dihydro-1-benzofuran-2-yl]-N-methylmethanamine;
1-[3-(1H-indol-1-yl)-2,3-dihydro-1-benzofuran-2-yl]-N-methyl methanamine;
7-fluoro-1-{7-fluoro-2-[(methylamino)methyl]-2,3-dihydro-1-benzofuran-3-yl}-3,3-dimethyl-1,3-dihydro-2H-indol-2-one;
1-[5-fluoro-3-(7-fluoro-3,3-dimethyl-2,3-dihydro-1H-indol-1-yl)-2,3-dihydro-1-benzofuran-2-yl]-N-methylmethanamine;
1-{6-fluoro-2-[(methylamino)methyl]-2,3-dihydro-1-benzofuran-3-yl}-3,3-dimethyl-1,3-dihydro-2H-indol-2-one;
3,3-dimethyl-1-{2-[(methylamino)methyl]-2,3-dihydro-1-benzofuran-3-yl}-1,3-dihydro-2H-indol-2-one;
1-[3-(3,4-dihydroquinolin-1(2H)-yl)-2,3-dihydro-1-benzofuran-2-yl]-N-methylmethanamine;
N-methyl-2-[(methylamino)methyl]-N-phenyl-2,3-dihydro-1-benzofuran-3-amine;
4-Fluoro-3-{6-fluoro-2-[(methylamino)methyl]-2,3-dihydro-1-benzofuran-3-yl}-1-isopropyl-1,3-dihydro-2H-benzimidazol-2-one;
4-Fluoro-3-{6-fluoro-2-[(methylamino)methyl]-2,3-dihydro-1-benzofuran-3-yl}-1-isopropyl-1,3-dihydro-2H-benzimidazol-2-one;
4-Fluoro-3-{6-fluoro-2-[(methylamino)methyl]-2,3-dihydro-1-benzofuran-3-yl}-1-isopropyl-1,3-dihydro-2H-benzimidazol-2-one and
pharmaceutically acceptable salts thereof.

26. A compound according to claim 1, selected from the group consisting of:
1-[(2R,3S)-3-(7-fluoro-3,3-dimethyl-2,3-dihydro-1H-indol-1-yl)-2,3-dihydro-1-benzofuran-2-yl]-N-methylmethanamine;
1-[(2R,3S)-3-(5-fluoro-3,3-dimethyl-2,3-dihydro-1H-indol-1-yl)-2,3-dihydro-1-benzofuran-2-yl]-N-methylmethanamine;
1-[(2R,3S)-3-(7-fluoro-3,3-dimethyl-2,3-dihydro-1H-indol-1-yl)-2,3-dihydro-1-benzofuran-2-yl]-N,N-dimethylmethanamine;
1-[(2R,3S)-3-(7-fluoro-3,3-dimethyl-2,3-dihydro-1H-indol-1-yl)-2,3-dihydro-1-benzofuran-2-yl]methanamine;
1-[(2S,3R)-3-(7-fluoro-3,3-dimethyl-2,3-dihydro-1H-indol-1-yl)-2,3-dihydro-1-benzofuran-2-yl]-N-methylmethanamine;
N-{[(2S,3R)-3-(7-fluoro-3,3-dimethyl-2,3-dihydro-1H-indol-1-yl)-2,3-dihydro-1-benzofuran-2-yl]methyl}propan-2-amine;
1-[(2S,3R)-3-(7-fluoro-3,3-dimethyl-2,3-dihydro-1H-indol-1-yl)-2,3-dihydro-1-benzofuran-2-yl]-N,N-dimethylmethanamine;
N-{[(2S,3R)-3-(7-fluoro-3,3-dimethyl-2,3-dihydro-1H-indol-1-yl)-2,3-dihydro-1-benzofuran-2-yl]methyl}ethanamine;
1-[(2R,3S)-3-(2-ethoxyphenoxy)-2,3-dihydro-1-benzofuran-2-yl]-N-methylmethanamine;
1-[3-(2,3-dihydro-1H-indol-1-yl)-2,3-dihydro-1-benzofuran-2-yl]-N-methylmethanamine;
1-[3-(1H-indol-1-yl)-2,3-dihydro-1-benzofuran-2-yl]-N-methyl methanamine;
7-fluoro-1-{(2S,3R)-7-fluoro-2-[(methylamino)methyl]-2,3-dihydro-1-benzofuran-3-yl}-3,3-dimethyl-1,3-dihydro-2H-indol-2-one;
1-[(2R,3S)-5-fluoro-3-(7-fluoro-3,3-dimethyl-2,3-dihydro-1H-indol-1-yl)-2,3-dihydro-1-benzofuran-2-yl]-N-methylmethanamine;
1-{6-fluoro-2-[(methylamino)methyl]-2,3-dihydro-1-benzofuran-3-yl}-3,3-dimethyl-1,3-dihydro-2H-indol-2-one;
3,3-dimethyl-1-{2-[(methylamino)methyl]-2,3-dihydro-1-benzofuran-3-yl}-1,3-dihydro-2H-indol-2-one;
1-[3-(3,4-dihydroquinolin-1(2H)-yl)-2,3-dihydro-1-benzofuran-2-yl]-N-methylmethanamine;
N-methyl-2-[(methylamino)methyl]-N-phenyl-2,3-dihydro-1-benzofuran-3-amine;
4-Fluoro-3-{(2SR,3RS)-6-fluoro-2-[(methylamino)methyl]-2,3-dihydro-1-benzofuran-3-yl}-1-isopropyl-1,3-dihydro-2H-benzimidazol-2-one;
4-Fluoro-3-{(2S,3R)-6-fluoro-2-[(methylamino)methyl]-2,3-dihydro-1-benzofuran-3-yl}-1-isopropyl-1,3-dihydro-2H-benzimidazol-2-one;
4-Fluoro-3-{(2R,3S)-6-fluoro-2-[(methylamino)methyl]-2,3-dihydro-1-benzofuran-3-yl}-1-isopropyl-1,3-dihydro-2H-benzimidazol-2-one;
and
pharmaceutically acceptable salts thereof.

27. A compound according to claim 1,
wherein said pharmaceutically acceptable salt is a hydrochloride.

28. A composition, comprising:
a. at least one compound according to claim 1; and
b. at least one pharmaceutically acceptable carrier.

29. A method for treating or preventing a condition selected from the group consisting of a vasomotor symptom, sexual dysfunction, gastrointestinal disorder, genitourinary disorder, chronic fatigue syndrome, fibromyalgia syndrome, depression disorder, endogenous behavioral disorder, cognitive disorder, diabetic neuropathy, pain, and combinations thereof in a subject in need thereof, comprising the step of:
administering to said subject an effective amount of a compound according to claim 1 or pharmaceutically acceptable salt thereof.

30. A method according to claim 29,
wherein said vasomotor symptom is hot flush.

31. A method according to claim 29,
wherein said sexual dysfunction is desire-related or arousal-related.

32. A method according to claim 29,
wherein said gastrointestinal disorder or said genitourinary disorder is stress incontinence or urge incontinence.

33. A method according to claim 29,
wherein said condition is chronic fatigue syndrome.

34. A method according to claim 29,
wherein said condition is fibromyalgia syndrome.

35. A method according to claim 29,
wherein said condition is a depression disorder selected from the group consisting of major depressive disorder, generalized anxiety disorder, panic disorder, attention deficit disorder with or without hyperactivity, sleep disturbance, social phobia, and combinations thereof.

36. A method according to claim 29,
wherein said condition is diabetic neuropathy.

37. A method according to claim 29,
wherein said condition is pain.

38. A method according to claim 37,
wherein said pain is acute centralized pain, acute peripheral pain, or a combination thereof.

39. A method according to claim 37,
wherein said pain is chronic centralized pain, chronic peripheral pain, or a combination thereof.

40. A method according to claim 37,
wherein said pain is neuropathic pain, visceral pain, musculoskeletal pain, bony pain, cancer pain, inflammatory pain, or a combination thereof.

41. A method according to claim 40,
wherein said neuropathic pain is associated with diabetes, post traumatic pain of amputation, lower back pain, cancer, chemical injury, toxins, major surgery, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, viral infection, bacterial infection, metastatic infiltration, adiposis dolorosa, burns, central pain conditions related to thalamic conditions, or a combination thereof.

42. A method according to claim 41,
wherein said neuropathic pain is post-herpetic neuralgia.

43. A method according to claim 40,
wherein said visceral pain is associated with ulcerative colitis, irritable bowel syndrome, irritable bladder, Crohn's disease, rheumatologic (arthralgias), tumors, gastritis, pancreatitis, infections of the organs, biliary tract disorders, or a combination thereof.

44. A method according to claim 37,
wherein said pain is female-specific pain.

45. A process for the preparation of a compound of formula Ia 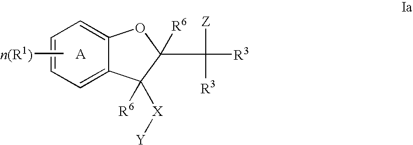
wherein:
z is OH or 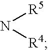
n is an integer from 0 to 4;
X is O, S, SO₂, or NR⁷;
Y is aryl substituted with 0-3 R¹ or heteroaryl substituted with 0-3 R¹;
R¹ is, independently at each occurrence, alkyl, alkoxy, halo, CF₃, OCF₃, arylalkoxy substituted with 0-3 R⁸, hydroxy, alkanoyloxy, nitro, nitrile, alkenyl, alkynyl, alkylsulfoxide, arylsulfoxide substituted with 0-3 R⁸, alkylsulfone, arylsulfone substituted with 0-3 R⁸, alkylsulfonamide, arylsulfonamide substituted with 0-3 R⁸, heteroarylsulfonamide substituted with 0-3 R⁸, heteroarylmethoxy substituted with 0-3 R⁸, alkylamido, or arylamido substituted with 0-3 R³; or
two adjacent R¹ also represent methylenedioxy;
each R³ is, independently at each occurrence, H, F, C₁-C₄ alkyl, or OR¹⁰;
R⁴ is H, C₁-C₄ alkyl, arylalkyl substituted with 0-3 R¹¹, heteroarylalkyl substituted with 0-3 R¹¹, cycloheptylmethyl, cyclohexylmethyl, cyclopentylmethyl, or cyclobutylmethyl; or
one R³ and R⁴, together with the nitrogen and carbon atoms through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃; or
R⁵ is H, C₁-C₄ alkyl, arylalkyl substituted with 0-3 R¹², heteroarylalkyl substituted with 0-3 R¹², cycloheptylmethyl, cyclohexylmethyl, cyclopentylmethyl, or cyclobutylmethyl; or
R⁴ and R⁵, together with the nitrogen through which they are attached, form a mono- or bi-cyclic ring of 3 to 7 ring atoms, where one carbon may be optionally replaced with N, O, S, or SO₂, and where any carbon ring atom or additional N atom may be optionally substituted with C₁-C₄ alkyl, F, or CF₃;
each R⁶ is, independently at each occurrence, H, alkyl, or perfluoroalkyl;
R⁷ is H, alkyl, or aryl substituted with 0-3 R¹³; or
R⁷ and Y, together with the nitrogen through which they are attached, form an aryl fused heterocycle, where one carbon may be optionally replaced with N, O, S, CO, or SO₂, and where any carbon ring atom or additional N atom may be optionally substituted with C₁-C₄ alkyl, F, or CF₃;
R⁸, R¹¹, R¹², and R¹³ are, independently at each occurrence, alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or
two adjacent R⁸, or two adjacent R¹¹, or two adjacent R¹², or two adjacent R¹³ also represent methylenedioxy;
R¹⁰ is H or C₁-C₄ alkyl; and
wherein
1-3 carbon atoms in ring A may optionally be replaced with N,
which process comprises subjecting a compound of formula II below to a ring-closing reaction to form the dihyrobenzofuran ring of formula Ia 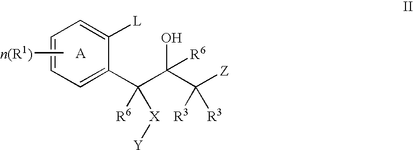
wherein
Z, n, X, Y, R¹, R³, R⁶ and A are as defined hereinabove for formula Ia and wherein L is a leaving group.

46. The process of claim 45, wherein the ring-closing reaction comprises an intramolecular nucleophilic replacement of leaving group L of the compound of formula II.

47. The process of claim 46, wherein the intramolecular nucleophilic replacement is performed in a solvent in the presence of a base.

48. The process of claim 45, wherein the ring-closing reaction comprises an intramolecular coupling reaction.

49. The process of claim 48, wherein the intramolecular coupling reaction is performed in the presence of a transition metal catalyst and a phosphine ligand.

50. The process of claim 48, wherein the intramolecular coupling reaction is performed in a solvent in the presence of a base.

51. The process of claim 45, wherein Z of the compound of formula II is OH and wherein the process further comprises converting Z into a leaving group and displacing the leaving group with 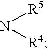
wherein R⁴ and R⁵ are as defined hereinabove for formula Ia.

52. The process of claim 45, wherein the compound of formula II is formed by opening the epoxide ring of a compound of formula III below 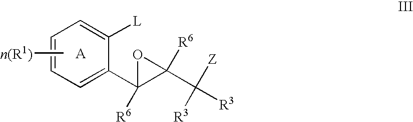
wherein
Z, n, R¹, R³, R⁶ and A are as defined hereinabove for formula Ia and
wherein
L is a leaving group.

53. The process of claim 52, wherein the epoxide ring opening comprises reacting a suitably nucleophilic compound of formula XY with the epoxide compound of formula Ill.

54. The process of claim 53, wherein the compound of formula XY is treated with a base prior to or during its reaction with the epoxide compound of formula III.

55. The process of claim 53, wherein the epoxide compound of formula III is treated with a Lewis acid prior to or during its reaction with the compound of formula XY.

56. The process of claim 52, wherein the epoxide ring opening is performed in the presence of a solvent.
